SEQUENCE LISTING (20 sequences)

SEQ ID NO: 1 - protein, 99 aa, Artificial Sequence
  Description of Artificial Sequence Synthetic polypeptide
RRVGDELEKEYERAFSSFSAQLHVTPTTARELFGQVATQLFSDGNINWGRVVALFSFGGFLALKLVDKELEDLVSRLASFLSEFLAKTLANWLRENGGW

SEQ ID NO: 2 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 3 - protein, 25 aa, Homo sapiens
TGYDNREIVMKYIHYKLSQRGYEWD

SEQ ID NO: 4 - protein, 175 aa, Homo sapiens
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSGWMTFLEVTGKICEMLSLLKQYC

SEQ ID NO: 5 - protein, 30 aa, Homo sapiens
FGYIYRLAQDYLQCVLQIPQPGSGPSKTSR

SEQ ID NO: 6 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 7 - protein, 27 aa, Homo sapiens
MSQSNRELVVDFLSYKLSQKGYSWSQF

SEQ ID NO: 8 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETRLADWIHSSGGWAEFTALYGDGALEEARRLREGNWASVRTVLTGAVALGALVTVGAFFASK

SEQ ID NO: 9 - protein, 24 aa, Homo sapiens
SAPDTRALVADFVGYKLRQKGYVC

SEQ ID NO: 10 - protein, 350 aa, Homo sapiens [sequence omitted]

SEQ ID NO: 11 - protein, 20 aa, Homo sapiens
DLYRQSLEIISRYLREQATG

SEQ ID NO: 12 - protein, 24 aa, Artificial Sequence
  Description of Artificial Sequence Synthetic peptide
PRLDIRGLVVDYVTYKLSQNGYEW

SEQ ID NO: 13 - DNA, 720 bp, Homo sapiens
  CDS (1)..(717)
atggcgcacgctgggagaagtggttacgataaccgggagatagtgatgaagtacatccattataagctgtcgcagaggggctacgagtgggatgcgggagatgtgggcgccgcgcccccgggggccgcccccgcaccgggcttcttctcctcccagcccgggcacacgccccatccagccgcatcccgggacccggtcgccaggacctcgccactacagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggatgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtga

SEQ ID NO: 14 - protein, 239 aa, Homo sapiens [sequence omitted]

SEQ ID NO: 15 - protein, 222 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFHHHHHHHHHH

SEQ ID NO: 16 - protein, 158 aa, Homo sapiens
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSHHHHHH

SEQ ID NO: 17 - protein, 25 aa, Artificial Sequence
  Description of Artificial Sequence Synthetic peptide
TGYDNREIVMKYIHYKLSQRGYEWD

SEQ ID NO: 18 - protein, 25 aa, Artificial Sequence
  Description of Artificial Sequence Synthetic peptide
FGYIYRLAQDYLQCVLQIPZPGSGP

SEQ ID NO: 19 - protein, 27 aa, Artificial Sequence
  Description of Artificial Sequence Synthetic peptide
MSQSNRELVVDFLSYKLSQKGYSWSQF

SEQ ID NO: 20 - protein, 25 aa, Artificial Sequence
  Description of Artificial Sequence Synthetic peptide
TWHMYGNQRDYIGDRSKIVYKLEYE

(19) United States
(12) Patent Application Publication
(10) Pub. No.: US 2011/0263510 A1
(43) Pub. Date: Oct. 27, 2011

(54) Methods of Inhibiting Cell Death or Inflammation in a Mammal

(76) Inventors: John M. Harlan, Seattle, WA (US); Robert K. Winn, Bainbridge Island, WA (US)

(21) Appl. No.: 12/514,436

(22) PCT Filed: Nov. 8, 2007
(86) PCT No.: PCT/US07/84052

§ 371 Date: May 12, 2011

Related U.S. Application Data
(60) Provisional application No. 60/857,913, filed on Nov. 10, 2006.

(51) Int. Cl. A61K 38/17; A61P 43/00; A61K 38/10

(52) U.S. Cl. 514/18.9

(57) ABSTRACT

Methods are provided for inhibiting cell death or inflammation in a mammal concurrent with or after the onset of a condition expected to lead to cell death or inflammation. The methods each include the step of administering to a mammal a Bcl protein in an amount sufficient to inhibit cell death or inflammation in the mammal. Methods are also provided for identifying a Bcl protein that inhibits cell death or inflammation when administered to a mammal concurrent with or after the onset of a condition expected to lead to cell death or inflammation.

### CROSS REFERENCE TO RELATED APPLICATIONS

This application claims priority under 35 U.S.C. §119(e) from U.S. Provisional Patent Application No. 60/857,913, entitled “METHODS OF INHIBITING CELL DEATH OR INFLAMMATION IN A MAMMAL” filed on 10 Nov. 2006, which is incorporated herein by reference in its entirety.

### STATEMENT OF GOVERNMENT SUPPORT

This invention was made by government support by Grant Nos. NIH HL72262, GM66197 and GM42686 from the National Institutes of Health. The Government has certain rights in this invention.

### FIELD

The invention relates to the use of exogenously administered proteins (such as Bcl-2 proteins) to inhibit cell death and/or inflammation concurrent with or after the onset of a condition expected to lead to cell death or inflammation.

### BACKGROUND

Programmed cell death is a normal and necessary part of mammalian development. For example, the development of separate fingers in a human fetus requires the programmed cell death of tissue between the developing fingers. The biochemical processes that cause programmed cell death can be triggered, however, by a variety of diseases and injuries. For example, programmed cell death can be triggered by traumatic injury, stroke, myocardial infarction, organ transplantation, and mesenteric and peripheral vascular disease. The programmed cell death further undermines the health of the injured or diseased organism.

Each of the foregoing types of diseases and injuries typically include some ischemia and reperfusion injury, which occurs when previously interrupted blood flow is restored to living tissue. For example, blockage of a coronary artery can cause cardiac muscle death due to the temporary lack of blood supply to the cardiac tissue. Additional muscle can die when blood flow is restored to the cardiac muscle by the administration of thrombolytic drugs.

Chronic and acute inflammation can also damage or kill living cells in a mammal. For example, the inflammation associated with emphysema causes lung damage over time. Inflammation can trigger programmed cell death, or can damage living tissue by some other mechanism. Accordingly, there is a continuing need for methods and compositions for inhibiting cell death and/or inflammation in a mammal concurrent with or after the onset of a condition expected to lead to cell death or inflammation.

### SUMMARY

Methods for inhibiting cell death and/or inflammation in a mammal concurrent with or after the onset of a condition expected to lead to cell death or inflammation are provided, wherein the methods each include the step of administering to a mammal a Bcl protein in an amount sufficient to inhibit cell death and/or inflammation in the mammal concurrent with or after the onset of a condition expected to lead to cell death or inflammation. The methods can be used, for example, to treat injuries or diseases, in a mammal, that involve cell death (e.g., ischemia-reperfusion injury), and/or to treat injuries or diseases, in a mammal, that involve inflammation (e.g., asthma). The methods of this aspect of the invention can also be used, for example, to prophylactically treat a mammal to prevent or delay the onset of cell death and/or inflammation.

In another aspect, the invention provides methods for identifying a Bcl protein that inhibits cell death and/or inflammation when administered to a mammal concurrent with or after the onset of a condition expected to lead to cell death or inflammation, wherein the methods of this aspect of the invention each include the step of screening a plurality of proteins to identify a Bcl protein that inhibits cell death and/or inflammation when administered to a mammal concurrent with or after the onset of a condition expected to lead to cell death or inflammation. The methods of this aspect of the invention can be used, for example, to identify Bcl proteins that inhibit cell death and/or inflammation when administered to a mammal concurrent with or after the onset of a condition expected to lead to cell death or inflammation, and that can be used to treat injuries or diseases, in a mammal, that involve cell death and/or inflammation concurrent with or after the onset of a condition expected to lead to cell death or inflammation.

In a further aspect, the present invention provides methods for identifying a Bcl protein that inhibits cell death and/or inflammation when administered to a mammal concurrent with or after the onset of a condition expected to lead to cell death or inflammation, wherein the methods of this aspect of the invention each include the step of analyzing data obtained from an experiment wherein a plurality of proteins are screened to identify a Bcl protein that inhibits cell death and/or inflammation in a mammal concurrent with or after the onset of a condition expected to lead to cell death or inflammation. The methods of this aspect of the invention can be used, for example, to identify Bcl proteins that inhibit cell death and/or inflammation when administered to a mammal concurrent with or after the onset of a condition expected to lead to cell death or inflammation, and that can be used to treat injuries or diseases, in a mammal, that involve cell death and/or inflammation concurrent with or after the onset of a condition expected to lead to cell death or inflammation.

### BRIEF DESCRIPTION OF THE DRAWINGS

FIG. 1 shows a bar chart of creatine kinase concentration in blood plasma of eleven transgenic mice that expressed exogenous human Bcl-2 (hBcl-2) in their myeloid cells (identified as Bcl-2 in FIG. 1), and a bar chart of creatine kinase concentration in blood plasma of nine non-transgenic, control, C57BL/6 mice that did not express exogenous hBcl-2 in their myeloid cells. Creatine kinase concentration was measured after the mice had been subjected to ischemia-reperfusion injury as described in Example 1. Creatine kinase concentration is expressed in units per liter (U/L) (*p<0.05).

FIG. 2 shows a bar chart of creatine kinase concentration in blood plasma of eight control C57BL/6 mice (identified as C57 in FIG. 2) that had suffered ischemia-reperfusion injury, and a bar chart of creatine kinase concentration in blood plasma of eight EμT-Bcl-2 mice (identified as EmBcl-2 (T-cell) in FIG. 2) that had suffered ischemic injury. (*p<0.05).

FIG. 3 shows a bar chart of the percentage of TUNEL positive cells in muscle tissue from the legs of six control C57BL/6 mice (abbreviated as C57), five EμT-Bcl-2 mice (abbreviated as EμT) that express hBcl-2 in their T-cells, six EμB-Bcl-2 mice (abbreviated as EμB) that express hBcl-2 in their B-cells, and five hMRP8-myeloid-Bcl-2 mice (abbreviated as hMRP) that express hBcl-2 in their myeloid cells. As described in Example 3, all of the mice had suffered ischemia-reperfusion injury (*p<0.05 versus C57).

FIG. 4 shows a bar chart of creatine kinase concentration, after ischemia and reperfusion, in blood plasma of six mice (identified as tg+ mice) that had received an injection, before ischemia, of blood plasma extracted from mice that express hBcl-2 in their T-lymphocytes; and a bar chart of creatine kinase concentration, after ischemia and reperfusion, in blood plasma of mice (identified as tg− mice) that had received an injection, before ischemia, of blood plasma extracted from six littermate control mice that did not express hBcl-2 in their T-lymphocytes. (*p<0.05).

FIG. 5 shows a bar chart of creatine kinase concentration, after ischemia and reperfusion, in blood plasma of 12 mice (identified as rBcl-2) that had received an injection, before ischemia and reperfusion, of recombinant human Bcl-2 (1 μg per mouse); and a bar chart of creatine kinase concentration, after ischemia and reperfusion, in blood plasma of 12 control mice (identified as control) that had received an injection, before ischemia and reperfusion, of either recombinant human ubiquitin or the vehicle solution used for injection of recombinant Bcl-2. There was no difference in the creatine kinase concentration between these two types of controls, and so these control data were combined. The creatine kinase concentrations were significantly different at p<0.05.

FIG. 6 shows a bar chart of the infarct volume (V_{infarct}) as a percentage of the left ventricular volume (V_{LV}) for five hMRP8-Bcl-2 mice that express hBcl-2 in their myeloid cells (identified as Bcl-2/2 mice in FIG. 9) and five C57BL/6 control mice; a bar chart of the infarct volume (V_{infarct}) as a percentage of the area-at-risk volume (V_{AAR}) for C57BL/6 control mice and hMRP8-Bcl-2 mice; and a bar chart of the area-at-risk volume (V_{AAR}) as a percentage of the left ventricular volume (V_{LV}) for C57BL/6 control mice and hMRP8-Bcl-2 mice. V_{infarct}/V_{LV} and V_{infarct}/V_{AAR} was significantly different between Bcl-2/2 mice and C57BL/6 mice at p<0.05.

FIG. 7 shows a bar chart of the infarct volume (V_{infarct}) as a percentage of the left ventricular volume (V_{LV}) for six C57BL/6 control mice and four EμT-Bcl-2 mice (that express Bcl-2 in their T cells); a bar chart of the infarct volume (V_{infarct}) as a percentage of the area-at-risk volume (V_{AAR}) for C57BL/6 control mice and EμT-Bcl-2 mice; and a bar chart of the area-at-risk volume (V_{AAR}) as a percentage of the left ventricular volume (V_{IN}) for C57BL/6 control mice and EμT-Bcl-2 mice. V_{infarct}/V_{LV} and V_{infarct}/V_{AAR} was significantly different between EμT-Bcl-2 mice and C57BL/6 mice at p<0.05

FIG. 8 shows bar charts of the infarct volume (V_{infarct}) as a percentage of the left ventricular volume (V_{LV}), the infarct volume (V_{infarct}) as a percentage of the area-at-risk volume (V_{AAR}), and the area-at-risk volume (V_{AAR}) as a percentage of the left ventricular volume (V_{LV}), for seven C57BL/6 mice that received an injection (before being subjected to myocardial ischemia and reperfusion) of CD11b+ cells that express exogenous human Bcl-2 (these mice are identified as Bcl-2/2 in FIG. 10), and for six C57BL/6 control mice (identified as Littermate Tg−) that had received an infusion (before being subjected to myocardial ischemia and reperfusion) of CD11b+ cells (that did not express exogenous Bcl-2) from their littermates. (*p<0.05)

FIG. 9 shows a survival curve for 12 mice that had been injected with recombinant human Bcl-2 (rBcl-2) prior to cecal ligation and puncture, and 12 control mice that were not injected with rBcl-2 prior to cecal ligation and puncture. The survival curves are significantly different at p<0.05.

FIG. 10 shows a bar chart of average tissue viability of skeletal muscle from 12 mice treated with exogenous recombinant human Bim (rhBim) or from 12 mice treated with exogenous recombinant human A1 (rhA1). Mice were subjected to ischemia as described and treated with either rhBim or rhA1 at the time of reperfusion. Tissue viability was measured 24 hours after reperfusion as described. (*p<0.05).

FIG. 11 shows a bar chart of average tissue viability of skeletal muscle from 11 mice treated with exogenous recombinant human Bim (rhBim) or from 12 mice treated with exogenous recombinant human A1 (rhA1). Mice were subjected to ischemia as described and treated with either rhBim or rhA1 4 hours after reperfusion. Tissue viability was measured 24 hours after reperfusion as described. (*p<0.05).

### DETAILED DESCRIPTION

### A. Introduction and General Overview

In one aspect, the present invention provides methods for inhibiting cell death in a mammal and/or inhibiting inflammation in a mammal concurrent with or after the onset of a condition expected to lead to cell death or inflammation. Each of the methods includes the step of administering to a mammal a Bcl protein in an amount sufficient to inhibit cell death and/or inflammation in the mammal concurrent with or after the onset of a condition expected to lead to cell death or inflammation.

The present invention provides a number of methods, reagents, and compounds that can be used for inhibiting cell death and/or inflammation concurrent with or after the onset of a condition expected to lead to cell death or inflammation.

It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

Inhibition of cell death in a mammal encompasses complete or partial inhibition of cell death in a mammal concurrent with or after the onset of a condition expected to lead to cell death. Inhibition of inflammation in a mammal encompasses complete or partial inhibition of inflammation in a mammal concurrent with or after the onset of a condition expected to lead to inflammation.

The methods of the present invention can be practiced on any mammal, such as primates (e.g., human beings), mammals of the genus Canis (e.g., domestic dog), mammals of the genus Felis (e.g., domestic cat), cattle, sheep, horses, goats and pigs.

In the practice of the present invention one or more types of Bcl proteins can be administered to a mammal concurrent with or after the onset of a condition expected to lead to cell death (e.g., suffering from a disease that causes cell death, or undergoing a medical treatment that causes cell death, or suffering from an injury that causes cell death). Examples of diseases, or medical treatments, that cause cell death include stroke, myocardial infarction, cardiac arrest, acute coronary syndrome/unstable angina, cardio-pulmonary by-pass grafting, traumatic shock, organ transplantation, mesenteric, retinal, and peripheral vascular disease, burns, frostbite, re-plantation of limbs and digits, traumatic brain injury, status epilepticus, Parkinson's disease, Huntington's disease, amyotrophic lateral sclerosis, Alzheimer's disease, macular degeneration, acute intracranial hemorrhage, acute renal failure, acute renal failure induced by shock, bacterial toxins, contrast dye or trauma, acute lung injury/adult respiratory distress syndrome, sepsis, meningitis, acute ischemic or alcoholic liver injury, Sjogren's disease, radiation-induced enteritis, and radiation-induced marrow failure.

### B. Exemplary Indications, Conditions, Diseases And Disorders

In the practice of the present invention one or more types of Bcl proteins can be administered to a mammal concurrent with or after the onset of a condition expected to lead to inflammation (e.g., suffering from an inflammatory disease, or suffering from an injury that causes inflammation, or undergoing a medical treatment that causes inflammation). Examples of inflammatory diseases include asthma, Crohn's disease, ulcerative colitis, hepatitis (e.g., viral chronic hepatitis), psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, chronic obstructive pulmonary disease, post-influenza pneumonia, SARS, tuberculosis, malaria, sepsis, cerebral malaria, Chagas disease, schistosomiasis, bacteria and viral meningitis, cystic fibrosis, multiple sclerosis, Alzheimer' disease, encephalomyelitis, sickle cell anemia, pancreatitis, transplantation (e.g., host-mediated rejection of transplanted tissue such as hematopoietic stem cells or an organ, graft mediated host response, such as graft vs. host disease), systemic lupus erythematosis, autoimmune diabetes, thyroiditis, and radiation pneumonitis.

Additionally, in the practice of the present invention one or more Bcl proteins can be administered to a mammal that is not suffering from an inflammatory disease or a disease associated with cell death. For example, one or more types of Bcl proteins can be administered prophylactically to a mammal to prevent, or decrease the likelihood of, the onset of cell death or inflammation, or to reduce the severity of cell death and/or inflammation that can subsequently occur. The mammal can be suffering from a disease that can cause cell death and/or inflammation, and the Bcl protein is administered to prevent, or decrease the likelihood of, the onset of cell death or inflammation, or to reduce the severity of cell death and/or inflammation that can subsequently occur. For example, the following categories of human patients can benefit from administration of Bcl to prevent, or decrease the likelihood of, the onset of cell death or inflammation: patients who suffer from transient ischemic attacks at risk for stroke, patients with unstable angina at risk for myocardial infarction, patients with trauma or burns at risk for multiple organ dysfunction, and patients undergoing cardio-pulmonary by-pass grafting at risk for post-operative organ dysfunction.

“Treating” or “treatment” includes the administration of the compositions, compounds or agents of the present invention to prevent or delay the onset of the symptoms, complications, or biochemical indicia of a disease, alleviating or ameliorating the symptoms or arresting or inhibiting further development of the disease, condition, or disorder (e.g., an inflammatory-related disease or disorder or a disease or disorder associated with cell death). “Treating” further refers to any indicia of success in the treatment or amelioration or prevention of the disease, condition, or disorder (e.g., an inflammatory-related disease or disorder or a disease or disorder associated with cell death), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with an inflammatory-related disease or disorder. The term “therapeutic effect” refers to the reduction, elimination, or prevention of the disease or disorder, symptoms of the disease or disorder, or side effects of the disease or disorder in the subject. “Treating” or “treatment” using the methods of the present invention includes preventing the onset of symptoms in a subject that can be at increased risk of an inflammatory-related disease or disorder or a disease or disorder associated with cell death but does not yet experience or exhibit symptoms, inhibiting the symptoms of an inflammatory-related disease or disorder or a disease or a disorder associated with cell death (slowing or arresting its development), providing relief from the symptoms or side-effects of an inflammatory-related disease or disorder or a disorder associated with cell death (including palliative treatment), and relieving the symptoms of an inflammatory-related disease or disorder or disorder associated with cell death (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

The terms “treatment”, “treating”, and the like are therefore used herein to refer to any treatment of any disease or condition in a mammal, e.g., particularly a human being, and includes: a) preventing a disease, condition, or symptom of a disease that causes cell death, or undergoing a medical treatment that causes cell death, or suffering from an injury that causes cell death from occurring in a subject which may be predisposed to the disease but has not yet been diagnosed as having it and/or that causes an inflammatory disease, or suffering from an injury that causes inflammation, or undergoing a medical treatment that causes inflammation; b) inhibiting a disease, condition, or symptom of a disease or condition that causes cell death, or undergoing a medical treatment that causes cell death, or suffering from an injury that causes cell death from occurring in a subject which may be predisposed to the disease but has not yet been diagnosed as having it and/or that causes an inflammatory disease, or suffering from an injury that causes inflammation, or undergoing a medical treatment that causes inflammation (e.g., arresting its development and/or delaying its onset or manifestation in the patient; and/or c) relieving a disease, condition, or symptom of a disease or condition that causes cell death, or undergoing a medical treatment that causes cell death, or suffering from an injury that causes cell death from occurring in a subject which may be predisposed to the disease but has not yet been diagnosed as having it and/or that causes an inflammatory disease, or suffering from an injury that causes inflammation, or undergoing a medical treatment that causes inflammation, e.g., causing regression of the condition or disease and/or its symptoms. A chronic disease or condition is a disease or condition that is long-lasting or recurrent. The term chronic describes the course of the disease, or its rate of onset and development. A chronic course is distinguished from a recurrent course; recurrent diseases or conditions relapse repeatedly, with periods of remission in between. Treatment of recurrent diseases and conditions with the Bcl proteins disclosed herein are also contemplated. A chronic disease or condition can have one or more of the following characteristics: a chronic disease or condition is permanent, leaves residual disability, can be caused by nonreversible pathological alteration, requires special training of the patient for rehabilitation, or can be expected to require a long period of supervision, observation, or care.

“Concomitant administration” of a known drug or agent with a Bcl protein of the present invention means administration of the drug and the Bcl protein at such time that both the known drug and the compound will have a therapeutic effect or diagnostic effect. Such concomitant administration can involve concurrent (i.e., at the same time), prior, or subsequent administration of the drug with respect to the administration of the Bcl protein of the present invention. A person of ordinary skill in the art would have no difficulty determining the appropriate timing, sequence and dosages of administration for particular drugs and Bcl proteins of the present invention.

“Inhibitors,” and “modulators” of cell death and inflammation-related diseases and disorders (complete or partial inhibition of cell death in a mammal concurrent with or after the onset of cell death or complete or partial inhibition of inflammation in a mammal concurrent with or after the onset of inflammation), e.g., Bcl proteins, are used to refer to inhibitory or modulating molecules, respectively, identified herein. “Modulator” includes inhibitors and activators. Inhibitors are agents that, e.g., bind to, partially or totally block stimulation, decrease, prevent, delay activation, inactivate, desensitize, or down regulate the cell death or inflammation.

“Biological activity” and “biologically active” are used in reference to complete or partial inhibition of cell death in a mammal concurrent with or after the onset of cell death or complete or partial inhibition of inflammation in a mammal concurrent with or after the onset of inflammation, e.g., by administrating an amount of a Bcl protein as described herein effective to inhibit cell death or inflammation in the mammal. Preferred biological activities include the ability to inhibit cell death concurrent with or after the onset of cell death or treat an inflammatory-related disease or disorder concurrent with or after the onset of the inflammatory-related disease or disorder. Accordingly, the administration of the Bcl proteins or agents of the present invention can prevent or delay, to alleviate, or to arrest or inhibit development of the symptoms or conditions associated with cell death or an inflammatory-related disease or disorder.

### C. Exemplary Therapeutic Agents

“Bcl protein” refers to a protein that inhibits cell death in a mammal when administered to the mammal concurrent with or after the onset of a condition expected to lead to cell death, and/or inhibits inflammation in a mammal when administered to the mammal concurrent with or after the onset of a condition expected to lead to inflammation, and that is a member of at least one of the following groups of proteins (identified as Groups (a) through (g)).

Group (a): A protein that includes an amino acid sequence that is at least 35% identical (e.g., at least 40%, or at least 45%, or at least 50%, or at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical)) to the amino acid sequence set forth in SEQ ID NO:1:

(SEQ ID NO: 1)
RRVGDELEKEYERAFSSFSAQLHVTPTTARELFGQVATQLFSDGNI
NWGRVVALFSFGGFLALKLVDKELEDLVSRLASFLSEFLAKTLANW
LRENGGW.

The amino acid sequence set forth in SEQ ID NO:1 is a consensus sequence for the Bcl domain for members of the Bcl-2 family of proteins.

Group (b): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of the Bcl-2 protein consisting of the amino acid sequence set forth in SEQ ID NO:2 (GenBank accession number AAH27258). In some aspects, the protein is at least 50% similar to the following segment of Bcl-2 protein: TGYDNREIVMKYIHYKLSQRGYEWD (SEQ ID NO:3). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-2 protein consisting of the amino acid sequence set forth in SEQ ID NO:2. The Bcl-2 class of proteins are intracellular cytoplasmic proteins that inhibit cell death (see, e.g., J. M. Adams and S. Cory, Science 281:1322-1326 (Aug. 28, 1998); S. Cory, et al., Oncogene 22:8590-8607, 2003).

Group (c): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of an A1 protein (also referred to as a Bfl-1 protein), wherein the A1 protein consists of the amino acid sequence set forth in SEQ ID NO:4 (GenBank accession number AAC50438). In some aspects, the protein is at least 50% similar to the following segment of A-1 protein: FGYIYRLAQDYLQCVLQIPQPGSGPSKTSR (SEQ ID NO:5). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the A-1 protein consisting of the amino acid sequence set forth in SEQ ID NO:4. A-1 proteins are homologs of Bcl-2 and are intracellular cytoplasmic proteins that inhibit apoptosis (see, e.g., A. Karsan, et al., Blood 87(8):3089-3096, Apr. 15, 1996; S. S. Choi et al., Mammalian Genome 8:781-782, 1997).

Group (d): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of a Bcl-X protein, wherein the Bcl-X protein consists of the amino acid sequence set forth in SEQ ID NO:6 (GenBank accession number Q07817). In some aspects, the protein is at least 50% similar to the following segment of Bcl-X protein: MSQSNRELVVDFLSYKLSQKGYSWSQF (SEQ ID NO:7). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-X protein consisting of the amino acid sequence set forth in SEQ ID NO:6. Bcl-X proteins are homologs of Bcl-2 and are intracellular cytoplasmic proteins that inhibit apoptosis (see, e.g., L. H. Boise, et al., Cell 74(4):597-608, Aug. 27, 1993.

Group (e): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of a Bcl-W protein consisting of the amino acid sequence set forth in SEQ ID NO:8 (GenBank accession number AAB09055). In some aspects, the protein is at least 50% similar to the following segment of Bcl-W protein: SAPDTRALVADFVGYKLRQKGYVC (SEQ ID NO:9). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Bcl-W protein consisting of the amino acid sequence set forth in SEQ ID NO:8. Bcl-W proteins are homologs of Bcl-2, and are intracellular cytoplasmic proteins that inhibit apoptosis (see, e.g., L. Gibson, et al., Oncogene 13(4):665-675, Aug. 15, 1996).

Group (f): A protein that includes at least 12 amino acids, wherein the protein is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a segment of an Mcl-1 protein, wherein the Mcl-1 protein consists of the amino acid sequence set forth in SEQ ID NO:10 (GenBank accession number AAF64255). In some aspects, the protein is at least 50% similar to the following segment of Mcl-1 protein: DLYRQSLETISRYLREQATG (SEQ ID NO:11). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to a segment of the Mcl-1 protein consisting of the amino acid sequence set forth in SEQ ID NO:10. Mcl-1 proteins are homologs of Bcl-2 and are intracellular cytoplasmic proteins that inhibit apoptosis.

Group (g): A protein that is at least 50% similar (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% similar) to a BH4 domain consisting of the following amino acid sequence: PRLDIRGLVVDYVTYKLSQNGYEW (SEQ ID NO:12). In some aspects, the protein is at least 50% identical (e.g., at least 55%, or at least 60%, or at least 65%, or at least 70%, or at least 75%, or at least 80%, or at least 85%, or at least 90%, or at least 95%, or at least 96%, or at least 97%, or at least 98%, or at least 99% identical) to the BH4 domain consisting of the amino acid sequence set forth in SEQ ID NO:12. BH4 (Bcl-2 homology domain 4) is an N-terminal domain found in Bcl-2, Bcl-X, and Bcl-W proteins. The amino acid sequence set forth in SEQ ID NO:12 is a consensus sequence for the BH4 domain of the Bcl-2 family of proteins.

Anti-apoptotic Bcl-2 family proteins containing a BH4-domain or BH4-like domain, comprising Class I (Bcl-2, Bcl-Xl), Class II (Bcl-W), and Class III (A1/Bfl-1, also referred to as BCL2A1; Orf-16; KS-Bcl-2; LMW5-HL; EBV-BHrfl; and nr-13) proteins as defined by Zhang et al (J Biol Chem, 275:11092, 2000). The Mcl-1 is an anti-apoptotic member that does not have a BH4-like domain on solution structure though it has an alpha1 helix (Day et al, J Biol Chem 280:4738, 2005).

A “protein” is a molecule having a sequence of amino acids that are linked to each other in a linear molecule by peptide bonds. As used herein, the term “protein” includes proteins having at least 12 amino acids.

As used herein in connection with proteins useful in the practice of the present invention, the term “segment” refers to at least 12 contiguous amino acids, and can include the complete amino acid sequence of a protein.

Sequence identity (typically expressed as percent identity) in the context of two protein sequences refers to the number of amino acid residues in the two sequences that are the same when the two sequences are aligned for maximum correspondence over a specified comparison window (e.g., if two protein sequences are aligned, each protein has 100 amino acids, and 75 of the amino acids in the first sequence are the same as, and align with, 75 of the amino acids in the second sequence, then the percent identity is 75%). Sequence identity values provided herein refer to the value obtained using GAP (e.g., GCG programs (Accelrys, Inc., San Diego, Calif.) version 10) using the following parameters: percent identity using GAP Weight of 50 and Length Weight of 3. The entire amino acid sequence of a candidate protein and a reference protein are compared. GAP uses the algorithm of Needleman & Wunsch J. Mol. Biol. 48:443-53, 1970, to find the alignment of two complete sequences that maximizes the number of matches and minimizes the number of gaps. An equivalent method to GAP can be used. The term “equivalent method” refers to any sequence comparison method, such as a sequence comparison program, that, for any two sequences in question, generates an alignment having identical amino acid residue matches and an identical percent sequence identity when compared to the corresponding alignment generated by GAP

Programs for searching for alignments are well known in the art, e.g., BLAST and the like. For example, if the target species is human, a source of such amino acid sequences or gene sequences (germline or rearranged antibody sequences) can be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain aspects, an acceptable range of homology is greater than about 50%. It should be understood that target species can be other than human.

Sequence similarity is a statistical measure of the degree of relatedness of two compared protein sequences. The percent similarity is calculated by a computer program that assigns a numerical value to each compared pair of amino acids based on chemical similarity (e.g., whether the compared amino acids are acidic, basic, hydrophobic, aromatic, and the like) and/or evolutionary distance as measured by the minimum number of base pair changes that would be required to convert a codon encoding one member of a pair of compared amino acids to a codon encoding the other member of the pair. Calculations are made after a best fit alignment of the two sequences has been made empirically by iterative comparison of all possible alignments. (see, e.g., Henikoff, S., and Henikoff, J. G., Proc. Nat'l Acad. Sci. USA 89:10915-10919, 1992). For example, sequence similarity can be determined using the ClustalW alignment program for full alignment, single CPU mode, using the GONNET matrix, a gap opening penalty of 100, a gap closing penalty of −1, a gap extending penalty of 0.2 and a gap separation penalty of 4. In the aligned sequences, similarity is defined as two amino acids being identical or having conserved substitutions or having semi-conserved substitutions. The ClustalW alignment program is available, for example, on the Internet at the web page of the European Bioinformatics Institute, Wellcome Trust Genome Campus, Hinxton, Cambridge, CB10 1SD, U.K.

Representative examples of amino acid sequences of Bcl-2 proteins, useful in the practice of the present invention, are set forth in the protein database accessible through the Entrez search tool of the National Center for Biotechnology Information, U.S. National Library of Medicine, 8600 Rockville Pike, Bethesda, Md. 20894, under the following accession numbers (the amino acid sequences of each of the identified Bcl-2 proteins are incorporated herein by reference): AAN17784.1; AAB17352.1; AAC53460.1; AAK15454.1; AAC53458.1; AAB17354.1; AAA82174.1; AAF88137.1; AAC72232.1; CAC10003.1; AAC53459.1; AAA19257.1; CAA57886.1; AAB17353.1; AAB96881.1; CAA58557.1; AAA82173.1; AAC15799.1; AAA51039.1; AAK15455.1; AAK31308.1; AAK31307.1; CAA80657.1; AAF89532.1; AAK31306.1; BAB85856.2; AAP35872.1; AAH19307.1; BAB71819.1; CAA80061.1; AAF33212.1; AAP36940.1; CAA04597.1; AAB07677.1; AAR92491.1; AAA37281.1; AAH68988.1; AAC60701.2; AAK92201.1; CAB92245.1; AAA37282.1; BAC33767.1; AAP47159.1; AAA77686.1; BAA01978.1; BAC37060.1; AAA77687.1; AAA53662.1; CAA29778.1; AAH27258.1; AAO26045.1; AAB53319.1; BAC24136.1; AAA35591.1; CAA78018.1; BAC81344.1; BAD05044.1; AAA51814.1; AAA51813.1; AAN03862.1; CAA57844.1; AAH40369.1; BAB28740.1; BAB62748.1; AAH44130.1; AAH71291.1; AAK81706.1; AAH74505.1; AAO64470.1; BAD32203.1; CAA57845.1; AAO64468.1; AAH74021.1; AAC64200.1; AAB86430.1; AAB09056.1; BAB29912.1; BAB23468.1; AAB09055.1; BAA19666.2; AAH73259.1; CAF93123.1; AAO13177.2; CAF96873.1; AAL35559.1; AAP21091.1; AAB97953.1; AAG02475.1; AAK55419.1; AAH27536.1; AAB97956.1; AAH28762.1; AAB97954.1; AAO89009.1; AAP35767.1; AAH16281.1; AAC50438.1; AAC50288.1; CAG46735.1; AAP36152.1; CAG02784.1; CAA70566.1; AAF89533.1; AAO22992.1; AAA03620.1; CAG46760.1; BAC40796.1; CAA73684.1; BAC53619.1; AAH55592.1; AAH66960.1; AAC48806.1; AAF71267.1; AAH04431.1; AAO74828.1; AAA93066.1; AAA74466.1; CAA58997.1; CAG33700.1; BAB85810.1; AAH14175.1; AAA03619.1; AAF98242.1; AAM74949.1; CAD10744.1; AAF71760.1; AAD13295.1; AAH78835.1; AAA75200.1; AAC60700.2; AAH53380.1; AAH18228.1; BAB28776.1; AAA03622.1; AAD31644.1; AAF36411.1; AAC26327.1; AAM34436.1; CAE54428.1; AAH03839.1; AAH21638.1; AAH05427.1; AAC31790.1; BAC77771.1; AAA74467.1; AAF64255.1; AAP36208.1; AAP35286.1; AAH71897.1; AAH17197.1; AAF74821.1; AAD13299.1; AAG00896.1; AAH78871.1; AAH30069.1; AAAC53582.1; AAB87418.1; BAC21258.1; AAF09129.1; AAH63201.1; AAK06406.1; AAR84081.1; AAP36565.1; AAP35936.1; AAH06203.1; AAD51719.1; AAD31645.1; and AAC50142.1.

The “amino acid” residues identified herein are in the natural L-configuration. In keeping with standard polypeptide nomenclature, J. Biol. Chem., 243:3557-59, (1969), abbreviations for amino acid residues are as shown in the following table.

| 1-Letter | 3-Letter | Amino Acid |
|---|---|---|
| Y | Tyr | L-tyrosine |
| G | Gly | L-glycine |
| F | Phe | L-phenylalanine |
| M | Met | L-methionine |
| A | Ala | L-alanine |
| S | Ser | L-serine |
| I | He | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gin | L-glutamine |
| E | Glu | L-glutamic acid |
| W | Trp | L-tryptohan |
| R | Arg | L-arginine |
| D | Asp | L-aspartic acid |
| N | Asn | L-asparagine |
| C | Cys | L-cysteine |

It should be noted that all amino acid residue sequences are represented herein by formulae whose left to right orientation is in the conventional direction of amino-terminus to carboxy-terminus.

### D. Bcl Peptides

The invention provides isolated Bcl peptides which inhibit cell death and/or inflammation concurrent with or after the onset of a condition expected to lead to cell death or inflammation. Exemplary Bcl peptides of the invention have an amino acid sequence including those described herein, and analogs, derivatives, amidated variations and conservative variations thereof, wherein the Bcl peptides inhibit cell death in a mammal and/or inhibit inflammation in a mammal concurrent with or after the onset of a condition expected to lead to cell death or inflammation. The Bcl peptides of the invention include those described herein, as well as the broader groups of peptides having hydrophilic and hydrophobic substitutions, and conservative variations thereof.

“Isolated” when used in reference to a peptide, refers to a peptide substantially free of proteins, lipids, nucleic acids, for example, with which it might be naturally associated. Those of skill in the art can make similar substitutions to achieve peptides with greater and a broader host range. For example, the invention includes the peptides described herein, as well as analogs or derivatives thereof, as long as the bioactivity (e.g., inhibition of cell death and/or inflammation concurrent with or after the onset of a cell death or inflammation event occurs) of the peptide remains. Minor modifications of the primary amino acid sequence of the peptides of the invention can result in peptides that have substantially equivalent activity as compared to the specific peptides described herein. Such modifications can be deliberate, as by site-directed mutagenesis, or can be spontaneous. All of the Bcl peptides produced by these modifications are included herein as long as the biological activity of the original Bcl peptide still exists.

Further, deletion of one or more amino acids can also result in a modification of the structure of the resultant molecule without significantly altering its biological activity. This can lead to the development of a smaller active molecule that would also have utility. For example, amino or carboxy terminal amino acids that can not be required for biological activity of the particular peptide can be removed. Peptides of the invention include any analog, homolog, mutant, isomer or derivative of the peptides disclosed in the present invention, so long as the bioactivity as described herein remains. All peptides can be synthesized using L amino acids, however, all D forms of the peptides can be synthetically produced. In addition, C-terminal derivatives can be produced, such as C-terminal methyl esters and C-terminal amidates, in order to increase the activity of a Bcl peptide of the invention. The peptide can be synthesized such that the sequence is reversed whereby the last amino acid in the sequence becomes the first amino acid, and the penultimate amino acid becomes the second amino acid, and so on. It is well known that such reversed peptides usually have similar activities to the original sequence.

In certain aspects, the peptides of the invention include peptide analogs and peptide mimetics. Indeed, the peptides of the invention include peptides having any of a variety of different modifications, including those described herein.

Peptide analogs of the invention are generally designed and produced by chemical modifications of a lead peptide, including, e.g., any of the particular peptides described herein, such as any of the following sequences disclosed in the tables. The present invention clearly establishes that these peptides in their entirety and derivatives created by modifying any side chains of the constituent amino acids have the ability to inhibit, prevent, or destroy the growth or proliferation of microbes such as bacteria, fungi, viruses, parasites or the like. The present invention further encompasses polypeptides up to about 50 amino acids in length that include the amino acid sequences and functional variants or peptide mimetics of the sequences described herein.

In another aspect, a peptide of the present invention is a pseudopeptide. Pseudopeptides or amide bond surrogates refers to peptides containing chemical modifications of some (or all) of the peptide bonds. The introduction of amide bond surrogates not only decreases peptide degradation but also can significantly modify some of the biochemical properties of the peptides, particularly the conformational flexibility and hydrophobicity.

To improve or alter the characteristics of polypeptides of the present invention, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the polypeptides of the present invention can be produced as multimers including dimers, trimers and tetramers. Multimerization can be facilitated by linkers, introduction of cysteines to permit creation of interchain disulphide bonds, or recombinantly though heterologous polypeptides such as Fc regions.

It is known in the art that one or more amino acids can be deleted from the N-terminus or C-terminus without substantial loss of biological function. See, e.g., Ron, et al., Biol. Chem., 268: 2984, 1993. Accordingly, the present invention provides polypeptides having one or more residues deleted from the amino terminus. Similarly, many examples of biologically functional C-terminal deletion mutants are known (see, e.g., Dobeli, et al., 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above can be included in the present invention. Thus, the invention further includes variations of the polypeptides which show substantial Bcl polypeptide activity (reduce the incidence, extent or severity of cell death or inflammation in a mammal comprising administering to the mammal concurrent with or after the onset of a condition expected to lead to cell death or inflammation). Such mutants include deletions, insertions, inversions, repeats, and substitutions selected according to general rules known in the art so as to have little effect on activity.

There are two main approaches for studying the tolerance of an amino acid sequence to change, see, Bowie, et al., Science, 247: 1306, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the polypeptide of the present invention can be, for example: (i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the polypeptides of the present invention can include one or more amino acid substitutions, deletions, or additions, either from natural mutations or human manipulation. As indicated, changes are preferably of a minor nature, such as conservative amino acid substitutions that do not significantly affect the folding or activity of the protein. The following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Furthermore, polypeptides of the present invention can include one or more amino acid substitutions that mimic modified amino acids. An example of this type of substitution includes replacing amino acids that are capable of being phosphorylated (e.g., serine, threonine, or tyrosine) with a negatively charged amino acid that resembles the negative charge of the phosphorylated amino acid (e.g., aspartic acid or glutamic acid). Also included is substitution of amino acids that are capable of being modified by hydrophobic groups (e.g., arginine) with amino acids carrying bulky hydrophobic side chains, such as tryptophan or phenylalanine. Therefore, a specific aspect of the invention includes polypeptides that include one or more amino acid substitutions that mimic modified amino acids at positions where amino acids that are capable of being modified are normally positioned. Further included are polypeptides where any subset of modifiable amino acids is substituted. For example, a polypeptide that includes three serine residues can be substituted at any one, any two, or all three of said serines. Furthermore, any polypeptide amino acid capable of being modified can be excluded from substitution with a modification-mimicking amino acid.

The present invention is further directed to fragments of the polypeptides of the present invention. More specifically, the present invention embodies purified, isolated, and recombinant polypeptides comprising at least any one integer between 6 and 504 (or the length of the polypeptides amino acid residues minus 1 if the length is less than 1000) of consecutive amino acid residues. Preferably, the fragments are at least 6, preferably at least 8 to 10, more preferably 12, 15, 20, 25, 30, 35, 40, 50 or more consecutive amino acids of a polypeptide of the present invention.

The present invention also provides for the exclusion of any species of polypeptide fragments of the present invention specified by 5′ and 3′ positions or sub-genuses of polypeptides specified by size in amino acids as described above. Any number of fragments specified by 5′ and 3′ positions or by size in amino acids, as described above, can be excluded.

In addition, it should be understood that in certain aspects, the peptides of the present invention include two or more modifications, including, but not limited to those described herein. By taking into the account the features of the peptide drugs on the market or under current development, it is clear that most of the peptides successfully stabilized against proteolysis consist of a mixture of several types of the above described modifications. This conclusion is understood in the light of the knowledge that many different enzymes are implicated in peptide degradation.

### E. Bcl Peptides, Peptide Variants, and Peptide Mimetics

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid mimetics refers to chemical compounds that have a structure that is different from the general chemical structure of an amino acid, but which functions in a manner similar to a naturally occurring amino acid. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings (See Table 6 below).

“Peptide” as used herein includes peptides that are conservative variations of those peptides specifically exemplified herein. “Conservative variation” as used herein denotes the replacement of an amino acid residue by another, biologically similar residue. Examples of conservative variations include, but are not limited to, the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acids, or glutamine for asparagine, and the like. Neutral hydrophilic amino acids that can be substituted for one another include asparagine, glutamine, serine and threonine. The term “conservative variation” also includes the use of a substituted amino acid in place of an unsubstituted parent amino acid provided that antibodies raised to the substituted polypeptide also immunoreact with the unsubstituted polypeptide. Such conservative substitutions are within the definition of the classes of the peptides of the invention. “Cationic” as is used to refer to any peptide that possesses sufficient positively charged amino acids to have a pI (isoelectric point) greater than about 9.0.

The biological activity of the peptides can be determined by standard methods known to those of skill in the art.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, e.g., a polypeptide fragment of an Bcl protein having Bcl biological activity as described herein (reduce the incidence, extent or severity of cell death or inflammation in a mammal comprising administering to the mammal concurrent with or after the onset of a condition expected to lead to cell death or inflammation).

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, or citrulline. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Histidine residue mimetics can be generated by reacting histidyl with, e.g., diethylprocarbonate or para-bromophenacyl bromide. Other mimetics include, e.g., those generated by hydroxylation of lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from an Bcl polypeptide having Bcl biological activity of the invention (reduce the incidence, extent or severity of cell death or inflammation in a mammal comprising administering to the mammal concurrent with or after the onset of a condition expected to lead to cell death or inflammation), resulting in modification of the structure of the polypeptide, without significantly altering its biological activity. For example, amino- or carboxyl-terminal, or internal, amino acids that are not required for Bcl biological activity can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205, 1998; Hruby, Curr. Opin. Chem. Biol. 1: 114, 1997; Ostergaard, Mol. Divers. 3: 17, 1997; Ostresh, Methods Enzymol. 267: 220, 1996. Modified peptides of the invention can be further produced by chemical modification methods, see, e.g., Belousov, Nucleic Acids Res. 25: 3440, 1997; Frenkel, Free Radic. Biol. Med. 19: 373, 1995; Blommers, Biochemistry 33: 7886, 1994.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems, Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3, 1997) and automated synthesis can be achieved, e.g., using the ABI 431 A Peptide Synthesizer (Perkin Elmer) in accordance with the instructions provided by the manufacturer.

Peptides of the invention can be synthesized by such commonly used methods as t-BOC or FMOC protection of alpha-amino groups. Both methods involve stepwise syntheses whereby a single amino acid is added at each step starting from the C terminus of the peptide (See, Coligan, et al., Current Protocols in Immunology, Wiley Interscience, 1991, Unit 9). Peptides of the invention can also be synthesized by the well known solid phase peptide synthesis methods described in Merrifield, J. Am. Chem. Soc. 85:2149, 1962, and Stewart and Young, Solid Phase Peptides Synthesis, (Freeman, San Francisco, 1969, pp. 27-62), using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with 1% acetic acid solution which is then lyophilized to yield the crude material. This can normally be purified by such techniques as gel filtration on Sephadex G-15 using 5% acetic acid as a solvent. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation.

Analogs, polypeptide fragment of Bcl proteins having Bcl biological activity as described herein (reduce the incidence, extent or severity of cell death or inflammation in a mammal comprising administering to the mammal concurrent with or after the onset of a condition expected to lead to cell death or inflammation), are generally designed and produced by chemical modifications of a lead peptide, including, e.g., any of the particular peptides described herein.

### F. Bcl Polypeptides and Functional Variants Thereof

“Polypeptide” includes proteins, fusion proteins, oligopeptides and polypeptide derivatives, with the exception that peptidomimetics are considered to be small molecules herein.

A “fusion protein” is a type of recombinant protein that has an amino acid sequence that results from the linkage of the amino acid sequences of two or more normally separate polypeptides.

A “protein fragment” is a proteolytic fragment of a larger polypeptide, which can be a protein or a fusion protein. A proteolytic fragment can be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” or “peptide” is a polypeptide having a short amino acid sequence (i.e., 2 to about 200 amino acids). An oligopeptide is generally prepared by chemical synthesis.

Bcl proteins include, for example, naturally-occurring Bcl proteins, synthetic Bcl proteins that can incorporate non-natural amino acids, and Bcl fusion proteins in which a protein, peptide, amino acid sequence, or other chemical structure, is attached to a portion (e.g., N-terminal or C-terminal) of a Bcl protein (discussed below). Representative examples of proteins or chemical structures that can be fused to a Bcl protein include: human serum albumin, an immunoglobulin, polyethylene glycol, or other protein or chemical structure that, for example, increases the serum half-life of the Bcl protein, or increases the efficacy of the Bcl protein, or reduces the immunogenicity of the Bcl protein.

As is explained in detail below, “polypeptide derivatives” include without limitation mutant polypeptides, chemically modified polypeptides, and peptidomimetics.

The Bcl polypeptides of this invention, including the analogs and other modified variants, can generally be prepared following known techniques. Preferably, synthetic production of the Bcl polypeptides of the invention can be according to the solid phase synthetic method. For example, the solid phase synthesis is well understood and is a common method for preparation of polypeptides, as are a variety of modifications of that technique. Merrifield, J. Am. Chem. Soc., 85: 2149, 1964; Stewart and Young, Solid Phase polypeptide Synthesis, Pierce Chemical Company, Rockford, Ill., 1984; Bodanszky and Bodanszky, The Practice of polypeptide Synthesis, Springer-Verlag, New York, 1984; Atherton and Sheppard, Solid Phase polypeptide Synthesis: A Practical Approach, IRL Press, New York, 1989. See, also, the specific method described in Example 1 below.

Alternatively, Bcl polypeptides of this invention can be prepared in recombinant systems using polynucleotide sequences encoding the polypeptides.

A “variant” or “functional variant” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention can contain more than one of the following modifications within the same polypeptide. Preferred polypeptide derivatives retain a desirable attribute, which can be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

A polypeptide having an amino acid sequence identical to that found in a protein prepared from a natural source is a “wild type” polypeptide. Functional variants of Bcl polypeptides can be prepared by chemical synthesis, including without limitation combinatorial synthesis.

Functional variants of Bcl polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences can comprise substitutions, deletions and additions of amino acids, with the proviso that such amino acids are naturally occurring amino acids.

Thus, subjecting a nucleic acid that encodes a polypeptide to mutagenesis is one technique that can be used to prepare functional variants of polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis can be randomly targeted (i.e., mutations can occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Bcl polypeptides having deletions or insertions of naturally occurring amino acids can be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences can be prepared by directed mutagenesis.

As contemplated by this invention, “polypeptide” includes those having one or more chemical modification relative to another polypeptide, i.e., chemically modified polypeptides. The polypeptide from which a chemically modified polypeptide is derived can be a wild type protein, a functional variant protein or a functional variant polypeptide, or polypeptide fragments thereof; an antibody or other polypeptide ligand according to the invention including without limitation single-chain antibodies, crystalline proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharma. Res. 10: 1268, 1993). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. 1993, cited above).

Different host cells will contain different post-translational modification mechanisms that can provide particular types of post-translational modification of a fusion protein if the amino acid sequences required for such modifications is present in the fusion protein. A large number (about 100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and can influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation can vary depending on which host cells are used to produce the fusion protein.

Another type of post-translation modification is the phosphorylation of a free hydroxyl group of the side chain of one or more Ser, Thr or Tyr residues, Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which can have a different chemical version of the methionine residue encoded by a start codon, and these will result in amino termini with different chemical modifications.

For example, many or most bacterial proteins are synthesized with an amino terminal amino acid that is a modified form of methionine, i.e., N-formyl-methionine (fMet). Although the statement is often made that all bacterial proteins are synthesized with an fMet initiator amino acid; although this can be true for E. coli, recent studies have shown that it is not true in the case of other bacteria such as Pseudomonas aeruginosa (Newton et al., J. Biol. Chem. 274: 22143, 1999). In any event, in E. coli, the formyl group of fMet is usually enzymatically removed after translation to yield an amino terminal methionine residue, although the entire fMet residue is sometimes removed (see Hershey, Chapter 40, “Protein Synthesis” in: Escherichia coli and Salmonella Typhimurium: Cellular and Molecular Biology, Neidhardt, Frederick C., Editor in Chief, American Society for Microbiology, Washington, D.C., 1987, Volume 1, pages 613-647, and references cited therein.). E. coli mutants that lack the enzymes (such as, e.g., formylase) that catalyze such post-translational modifications will produce proteins having an amino terminal fMet residue (Guillon et al., J. Bacteriol. 174: 4294, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23: 263, 1998; and Driessen et al., CRC Crit. Rev. Biochem. 18: 281, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Annu. Rev. Physiol. 50: 333, 1988, and Bradbury et al. Lung Cancer 14: 239, 1996. About 50% of known endocrine and neuroendocrine peptide hormones are alpha-amidated (Treston et al., Cell Growth Differ. 4: 911, 1993). In most cases, carboxy alpha-amidation is required to activate these peptide hormones.

### G. Bcl Polypeptide Mimetic

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself. For example, polypeptides can exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity (e.g., reduce the incidence, extent or severity of cell death or inflammation in a mammal comprising administering to the mammal concurrent with or after the onset of a condition expected to lead to cell death or inflammation) can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean, BioEssays, 16: 683, 1994; Cohen and Shatzmiller, J. Mol. Graph., 11: 166, 1993; Wiley and Rich, Med. Res. Rev., 13: 327, 1993; Moore, Trends Pharmacol. Sci., 15: 124, 1994; Hruby, Biopolymers, 33: 1073, 1993; Bugg et al., Sci. Am., 269: 92, 1993, all incorporated herein by reference).

Thus, through use of the methods described above, the present invention provides compounds exhibiting enhanced therapeutic activity in comparison to the polypeptides described above. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., Int. J. Polypeptide Protein Res. 41: 181, 1993, incorporated herein by reference). Thus, the amino acid sequences of these compounds can be identical to the sequences of their parent L-amino acid polypeptides, except that one or more of the peptide bonds are replaced by an isosteric pseudopeptide bond. Preferably the most N-terminal peptide bond is substituted, since such a substitution would confer resistance to proteolysis by exopeptidases acting on the N-terminus.

To confer resistance to proteolysis, peptide bonds can also be substituted by retro-inverso pseudopeptide bonds (Dalpozzo, et al., Int. J. Polypeptide Protein Res. 41: 561-566, incorporated herein by reference). According to this modification, the amino acid sequences of the compounds can be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid derivatives of polypeptides represent another form of modified polypeptides that retain the important structural determinants for biological activity, yet eliminate the peptide bonds, thereby conferring resistance to proteolysis (Simon, et al., Proc. Natl. Acad. Sci. USA, 89: 9367, 1992, and incorporated herein by reference). Peptoids are oligomers of N-substituted glycines. A number of N-alkyl groups have been described, each corresponding to the side chain of a natural amino acid.

The invention includes polynucleotides encoding peptides of the invention. Exemplary polynucleotides encode peptides including those listed in Table 1, and analogs, derivatives, amidated variations and conservative variations thereof, wherein the peptides inhibit cell death and/or inflammation. The peptides of the invention include those disclosed herein, as well as the broader groups of peptides having hydrophilic and hydrophobic substitutions, and conservative variations thereof.

### H. Cell Death and Inflammation Assays

The ability of a Bcl protein to inhibit cell death in a mammal, and/or to inhibit inflammation in a mammal concurrent with or after the onset of a condition expected to lead to cell death or inflammation, can be assessed, for example, in a mammal subjected to ischemia-reperfusion injury. As used herein, “determining,” “measuring,” and “assessing,” and “assaying” are used interchangeably and include both quantitative and qualitative determinations. For example, one or more of the following markers and/or assays can be used to assess the ability of a Bcl protein to inhibit cell death and/or inflammation in a mammal subjected to ischemia-reperfusion injury: 1. Inhibition of inflammation and/or cell death is shown by a reduction in creatine kinase concentration in the plasma or serum of a mammal after ischemia-reperfusion of skeletal muscle (see, e.g., Example 6 herein, and Iwata, A., et al., Blood 100:2077, 2002); 2. Inhibition of inflammation and/or cell death is shown by a reduction in infarct size following ischemia-reperfusion of mammalian heart or mammalian brain (see, e.g., Palazzo, A. J., Am. J. Physiol. 275:H2300, 1998; Piot, C, Circulation 96:1598, 1997); 3. Inhibition of inflammation and/or cell death is shown by a reduction in blood urea nitrogen (BUN) and/or creatine following ischemia-reperfusion of mammalian kidney (see, e.g., Daemen, M., J. Clin. Invest. 104:541, 1999; Vukicevic, S., J. Clin. Invest. 102:202, 1998); 4. Inhibition of inflammation and/or cell death is shown by a reduction in aspartate aminotransferase (AST) and/or alanine aminotransferase (ALT) following ischemia-reperfusion of mammalian liver (see, e.g., Cursio, R., FASEB J. 13:253, 1999); 5. Inhibition of inflammation and/or cell death is shown by an improvement in arterial oxygen content following ischemia-reperfusion of mammalian lung; 6. Inhibition of inflammation and/or apoptosis is shown by a reduction in lung edema following ischemia-reperfusion of mammalian lung (see, e.g., Kowalski, T. F., J. Appl. Physiol. 68:125, 1990; 7. Inhibition of cell death is shown by a reduction in markers of cell death (e.g., by reduced DNA strand-breaks assessed by terminal deoxynucleotidyl transferase end labeling (TUNEL), by reduced caspase activation, by increased expression of phosphatidyl serine on the cell surface, by decreased DNA ladder of 180-200 base pair following electrophoresis) in tissue (e.g., skeletal muscle, heart, brain, lung, intestine, kidney, or liver) subjected to ischemia-reperfusion injury (see, e.g., Iwata, et al., Blood 100:2077, 2002; Piot, C., Circulation 96:1598, 1997; Namura, S., J. Neurosci. 18:3659, 1998; Noda, T., Am. J. Physiol. 274:G270, 1998; and Cursio, R., FASEB J. 13:253, 1999). The viability of tissue subjected to I/R was measured using 3-(4,5-dimethylthiazol-2-yl)-2,5-diphenyl tetrazolium (MTT) (Ferrera et al., J Mol Cell Cardiol 25:1091, 1993). Viable mitochondria reduce MTT to a water-insoluble salt that is soluble in isopropanol and can be extracted by isopropanol. Live tissue with functional mitochondria will therefore have an increased optical density relative to dead tissue.

Inhibition of inflammation and/or cell death concurrent with or after the onset of a condition expected to lead to cell death or inflammation as a result of administration of a Bcl protein can also be assessed in a mammal subjected to sepsis (e.g., sepsis due to cecal ligation and puncture, sepsis due to bacterial pneumonia, sepsis due to bacterial peritonitis), or in a mammal receiving injections or infusions (e.g., injections or infusions into the peritoneum, injections or infusions into the lung, subcutaneous injections or infusions, intra-dermal injections or infusions) of substances that promote sepsis (e.g., lipopolysaccharide, bacterial lipoproteins, lipoteichoic acid). Inhibition of inflammation and/or cell death concurrent with or after the onset of a condition expected to lead to cell death or inflammation as a result of administration of a Bcl protein is indicated by increased survival in mammals following initiation of sepsis by the injection or infusion of substances that promote sepsis.

### I. Screening Assays for Bcl Protein Therapeutic Candidates

In another aspect, the present invention provides methods for identifying a Bcl protein that inhibits cell death and/or inflammation when administered to a mammal concurrent with or after the onset of a condition expected to lead to cell death or inflammation. The methods of this aspect of the invention each include the step of screening a plurality of proteins to identify a Bcl protein that inhibits cell death and/or inflammation when administered to a mammal concurrent with or after the onset of a condition expected to lead to cell death or inflammation.

In the practice of this aspect of the invention, at least two proteins are screened to identify a Bcl protein that inhibits cell death or inflammation when administered to a mammal concurrent with or after the onset of a condition expected to lead to cell death or inflammation. Thus, for example, between two and 100 proteins can be screened to identify a Bcl protein that inhibits cell death or inflammation when administered to a mammal concurrent with or after the onset of a condition expected to lead to cell death or inflammation; or, for example, from 100 to 500 proteins can be screened to identify a Bcl protein that inhibits cell death or inflammation when administered to a mammal concurrent with or after the onset of a condition expected to lead to cell death or inflammation; or, for example, from 100 to 1000 proteins can be screened to identify a Bcl protein that inhibits cell death or inflammation when administered to a mammal concurrent with or after the onset of a condition expected to lead to cell death or inflammation; or, for example, more than 1000 proteins can be screened to identify a Bcl protein that inhibits cell death or inflammation when administered to a mammal concurrent with or after the onset of a condition expected to lead to cell death or inflammation.

Any useful assay can be used to identify a protein that inhibits cell death and/or inflammation when administered to a mammal concurrent with or after the onset of a condition expected to lead to cell death or inflammation. For example, a useful assay can be an in vitro assay, or an in vivo assay, or an assay that includes an in vitro component and an in vivo component. Representative examples of useful assays include the assays described supra for assessing the ability of a Bcl protein to inhibit cell death in a mammal, and/or inhibit inflammation in a mammal subjected to ischemia-reperfusion injury.

In another aspect, the present invention provides methods for identifying a Bcl protein that inhibits cell death or inflammation when administered to a mammal concurrent with or after the onset of a condition expected to lead to cell death or inflammation. The methods of this aspect of the invention each include the step of analyzing data obtained from an experiment wherein a plurality of proteins are screened to identify a Bcl protein that inhibits cell death and/or inflammation when administered to a mammal concurrent with or after the onset of a condition expected to lead to cell death or inflammation. The analysis can include comparing the effect(s) of candidate Bcl proteins on inflammation and/or cell death in viva and/or in vitro, and comparing the effect(s) of the candidate proteins to the effects on inflammation and/or cell death of a non-Bcl protein, or a Bcl protein that has been modified (e.g., by site-directed mutagenesis) to be biologically inactive, or to some other control treatment. A statistically significant increase in the amount of inhibition of cell death or inflammation caused by the candidate Bcl protein, compared to the amount of inhibition of cell death and/or inflammation caused by the control treatment, indicates that the candidate Bcl protein inhibits cell death or inflammation. If desired, the candidate Bcl protein can be subjected to further study.

Any of the methods disclosed herein for screening a plurality of Bcl proteins to identify a Bcl protein that inhibits cell death or inflammation in a mammal concurrent with or after the onset of a condition expected to lead to cell death or inflammation can be used in this aspect of the invention.

In the practice of this aspect of the invention, the analyzed data are obtained from an experiment wherein a plurality of proteins is screened to identify a Bcl protein that inhibits cell death or inflammation when administered to a mammal concurrent with or after the onset of a condition expected to lead to cell death or inflammation. For example, between two and 100 proteins can be screened to identify a Bcl protein that inhibits cell death or inflammation when administered to a mammal concurrent with or after the onset of a condition expected to lead to cell death or inflammation; or, for example, from 100 to 500 proteins can be screened to identify a Bcl protein that inhibits cell death or inflammation when administered to a mammal concurrent with or after the onset of a condition expected to lead to cell death or inflammation; or, for example, from 100 to 1000 proteins can be screened to identify a Bcl protein that inhibits cell death or inflammation when administered to a mammal concurrent with or after the onset of a condition expected to lead to cell death or inflammation; or, for example, more than 1000 proteins can be screened to identify a Bcl protein that inhibits cell death or inflammation when administered to a mammal concurrent with or after the onset of a condition expected to lead to cell death or inflammation.

### J. Treatment Regimes

The invention provides pharmaceutical compositions comprising one or a combination of Bcl proteins, for example, formulated together with a pharmaceutically acceptable carrier. Some compositions include a combination of multiple (e.g., two or more) peptides of the invention.

As used herein “pharmaceutically acceptable carrier” includes any and all solvents, dispersion media, coatings, antibacterial and antifungal agents, isotonic and absorption delaying agents, and the like that are physiologically compatible. In one aspect, the carrier is suitable for parenteral administration. Alternatively, the carrier can be suitable for intravenous, intraperitoneal or intramuscular administration. In another aspect, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is compatible with the active compound, use thereof in the pharmaceutical compositions is contemplated. Supplementary active compounds can also be incorporated into the compositions.

A “pharmaceutically acceptable salt” refers to a salt that retains the desired biological activity of the parent compound and does not impart any undesired toxicological effects (See, e.g., Berge, et al., J. Pharm. Sci., 66: 1, 1977). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In prophylactic applications, pharmaceutical compositions or medicaments are administered to a patient susceptible to, or otherwise at risk of a disease or condition (i.e., as a result of bacteria, fungi, viruses, parasites or the like) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease or condition in an amount sufficient to cure, or at least partially arrest, the symptoms of the disease or condition (e.g., biochemical and/or histologic), including its complications and intermediate pathological phenotypes in development of the disease or condition. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if the response starts to wane.

The pharmaceutical composition of the present invention should be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound can be orally administered, for example, with an inert diluent or an assimilable edible carrier.

Pharmaceutical compositions of the invention also can be administered in combination therapy, i.e., combined with other agents. For example, where cell death is caused by sepsis or ischemia-reperfusion, the combination therapy can include a composition of the present invention with at least one agent or other conventional therapy.

K. Routes of Administration

A composition of the present invention can be administered by a variety of methods known in the art. The route and/or mode of administration vary depending upon the desired results. The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. The peptide of the invention can be administered parenterally by injection or by gradual infusion over time. The peptide can also be prepared with carriers that protect the compound against rapid release, such as a controlled release formulation, including implants, transdermal patches, and microencapsulated delivery systems. Further methods for delivery of the peptide include orally, by encapsulation in microspheres or proteinoids, by aerosol delivery to the lungs, or transdermally by iontophoresis or transdermal electroporation. To administer a peptide of the invention by certain routes of administration, it can be necessary to coat the compound with, or co-administer the compound with, a material to prevent its inactivation. The method of the invention also includes delivery systems such as microencapsulation of peptides into liposomes or a diluent. Microencapsulation also allows co-entrapment of Bcl molecules along with the antigens, so that these molecules, such as antibiotics, can be delivered to a site in need of such treatment in conjunction with the peptides of the invention. Liposomes in the blood stream are generally taken up by the liver and spleen. Pharmaceutically acceptable diluents include saline and aqueous buffer solutions. Liposomes include water-in-oil-in-water CGF emulsions as well as conventional liposomes (Strejan, et al., J. Neuroimmunol., 7: 27, 1984). Thus, the method of the invention is particularly useful for delivering Bcl peptides to such organs. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, Ed., 1978, Marcel Dekker, Inc., New York. Other methods of administration will be known to those skilled in the art.

Preparations for parenteral administration of a peptide of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions can also be administered with medical devices known in the art. For example, in a preferred aspect, a therapeutic composition of the invention can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

Pharmacological agents can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187, 1995; Tjwa, Ann. Allergy Asthma Immunol. 75:107, 1995).

For example, some aspects can also include at least one pharmacological agent in an aerosolized, atomized or nebulized vapor form, e.g., administrable via a metered dose device or nebulizer, and the like such that aspects also include aerosolizing, vaporing or nebulizing one or more pharmacological agents for administration to a subject. Accordingly, for administration to the upper (nasal) or lower respiratory tract by inhalation, one or more therapeutic compositions or agents of the invention can be conveniently delivered from a nebulizer or a pressurized pack or other convenient means of delivering an aerosol spray. Pressurized packs can comprise a suitable propellant such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent can also be administered via nose drops, a liquid spray, such as via a plastic bottle atomizer or metered-dose inhaler. Typical of atomizers are the Mistometer (Wintrop) and the Medihaler (Riker).

When the peptides of the present invention are administered as pharmaceuticals, to humans and animals, they can be given alone or as a pharmaceutical composition containing, for example, 0.01 to 99.5% (or 0.1 to 90%) of active ingredient in combination with a pharmaceutically acceptable carrier.

Again by way of representative example, Bcl protein can be introduced into an animal body by application to a bodily membrane capable of absorbing the protein, for example the nasal, gastrointestinal and rectal membranes. The protein is typically applied to the absorptive membrane in conjunction with a permeation enhancer. (See, e.g., V. H. L. Lee, Crit. Rev. Ther. Drug Carrier Syst. 5:69, 1988; V. H. L. Lee, J. Controlled Release 13:213, 1990; V. H. L. Lee, Ed., Peptide and Protein Drug Delivery, Marcel Dekker, New York, 1991; DeBoer, A. G., et al., J. Controlled Release 13:241, 1990). For example, STDHF is a synthetic derivative of fusidic acid, a steroidal surfactant that is similar in structure to the bile salts, and has been used as a permeation enhancer for nasal delivery. (Lee, W. A., Biopharm., Nov./Dec. 22, 1990).

Bcl protein can be introduced in association with another molecule, such as a lipid, to protect the protein from enzymatic degradation. For example, the covalent attachment of polymers, especially polyethylene glycol (PEG), has been used to protect certain proteins from enzymatic hydrolysis in the body and thus prolong half-life (Fuertges, F., et al., J. Controlled Release 11:139, 1990). Many polymer systems have been reported for protein delivery (Bae, Y. H., et al., J. Controlled Release 9:271, 1989; Hori, R., et al., Pharm. Res. 6:813, 1989; Yamakawa, I., et al., J. Pharm. Sci. 79:505, 1990; Yoshihiro, I., et al., J. Controlled Release 10:195, 1989; Asano, M., et al., J. Controlled Release 9:111, 1989; Rosenblatt, J., et al., J. Controlled Release 9:195, 1989; Makino, K., J. Controlled Release 2:235, 1990; Takakura, Y., et al., J. Pharm. Sci. 78:117, 1989; Takakura, Y., et al., J. Pharm. Sci. 78:219, 1989.).

Administration “in combination with” one or more further therapeutic agents includes simultaneous (concurrent) and consecutive administration in any order.

### L. Effective Dosages

The protective protein therapeutic agent (e.g., a Bcl protein or nucleic acid encoding a protective Bcl protein) is administered in a therapeutically effective amount. “Therapeutically effective amount” as used herein for treatment of cell death and inflammatory related diseases and conditions refers to the amount of peptide used that is of sufficient quantity to reduce the incidence, extent or severity of cell death or inflammation in a mammal comprising administering to the mammal concurrent with or after the onset of a condition expected to lead to cell death or inflammation an amount of a Bcl protein effective to attain said reduction. The dosage ranges for the administration of peptides are those large enough to produce the desired effect. The amount of peptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the nature of any concurrent treatment, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's (Remington's Pharmaceutical Science, Mack Publishing Company, Easton, Pa.); Egleton, Peptides 18: 1431, 1997; Langer, Science 249: 1527, 1990. The dosage regimen can be adjusted by the individual physician in the event of any contraindications.

Dosage regimens of the pharmaceutical compositions of the present invention are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the compounds of the invention employed in the pharmaceutical composition at levels lower than that required to achieve the desired therapeutic effect and gradually increase the dosage until the desired effect is achieved. In general, a suitable daily dose of a compound of the invention is that amount of the compound which is the lowest dose effective to produce a therapeutic effect. Such an effective dose generally depends upon the factors described above. It is preferred that administration be intravenous, intramuscular, intraperitoneal, or subcutaneous, or administered proximal to the site of the target. If desired, the effective daily dose of a therapeutic composition can be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

For any Bcl protein, the effective dose can be estimated initially in any appropriate animal model (e.g., primate, rats and guinea pigs and other laboratory animals). The animal model is also typically used to achieve a desirable concentration range and route of administration. Such information can then be used to determine useful doses and routes for administration in humans or other mammals.

Therapeutic efficacy and possible toxicity of Bcl proteins can be determined by standard pharmaceutical procedures in experimental animals (e.g., ED₅₀, the dose therapeutically effective in 50% of the population; and LD₅₀, the dose lethal to 50% of the population). The dose ratio between therapeutic and toxic effects is the therapeutic index, and it can be expressed as the ratio ED₅₀/LD₅₀. Bcl proteins, which exhibit large therapeutic indices, are preferred. The data obtained from animal studies is used in formulating a range of dosage for use in humans or other mammals. The dosage of such compounds lies preferably within a range of circulating concentrations that include the ED₅₀ with little or no toxicity. The dosage typically varies within this range depending upon the dosage form employed, sensitivity of the patient, and the route of administration.

The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Thus, the administration of the therapeutic agents in accordance with the present invention can be in a single dose, in multiple doses, in a continuous or intermittent manner, depending, for example, upon the recipient's physiological condition, whether the purpose of the administration is therapeutic or prophylactic, and other factors known to skilled practitioners. The administration of the agents of the invention can be essentially continuous over a preselected period of time or can be in a series of spaced doses. Both local and systemic administration is contemplated.

An effective dose of each of the Bcl peptides disclosed herein as potential therapeutics for use in treating cell death and inflammatory-related diseases and conditions is from about 1 μg to 500 mg/kg body weight, per single administration, which can readily be determined by one skilled in the art. As discussed above, the dosage depends upon the age, sex, health, and weight of the recipient, kind of concurrent therapy, if any, and frequency of treatment. Other effective dosage range upper limits are 100 mg/kg body weight, 50 mg/kg body weight, 25 mg/kg body weight, and 10 mg/kg body weight. Other exemplary dosages include administration of at least 50 ng/kg/day, such as from 50 ng/kg/day to 50 mg/kg/day, or such as from 0.5 mg/kg/day to 50 mg/kg/day, for a period of time sufficient to reduce the incidence, extent or severity of cell death or inflammation in a mammal concurrent with or after the onset of a condition expected to lead to cell death or inflammation.

Typically, the Bcl protein is administered to the mammal on multiple occasions (e.g., daily). For example, a Bcl protein can be administered to a mammal at least once per day for a period of from 1 day to 20 days, or from 1 day to 40 days, or from 1 day to 60 days to reduce the incidence, extent or severity of cell death or inflammation in a mammal concurrent with or after the onset of a condition expected to lead to cell death or inflammation. The Bcl protein can also be administered up to 1, 2, 3, 4, 5, 6, 7, 8, 9, 10, 11, 12, 24, or 48 hours after the onset of a condition expected to lead to cell death or inflammation. The Bcl protein can also be administered to the mammal intermittently or continuously for at least 12 or 24 or 48 hours or more after the onset of a condition expected to lead to cell death or inflammation. Bcl protein can be administered indefinitely to a mammalian subject to treat a chronic medical condition (e.g., at least once per day each day during the remaining lifetime of the recipient). As discussed above, the Bcl protein can also be administered with at least one additional agent. The abbreviation “ng” is an abbreviation for nanogram, or nanograms, as appropriate. The abbreviation “mg” is an abbreviation for milligram, or milligrams, as appropriate. The abbreviation “kg” is an abbreviation for kilogram, or kilograms, as appropriate.

Some compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, See, e.g., U.S. Pat. Nos. 4,522,811; 5,374,548; and 5,399,331. The liposomes can comprise one or more moieties which are selectively transported into specific cells or organs, thus enhance targeted drug delivery (See, e.g., Ranade, J. Clin. Pharmacol., 29: 685, 1989). Exemplary targeting moieties include folate or biotin (See; e.g., U.S. Pat. No. 5,416,016 to Low, et al.); mannosides (Umezawa, et al., Biochem. Biophys. Res. Commun., 153: 1038, 1988); antibodies (Bloeman, et al., FEBS Lett., 357: 140, 1995; Owais, et al., Antimicrob. Agents Chemother., 39: 180, 1995); surfactant protein A receptor (Briscoe, et al., Am. J. Physiol., 1233: 134, 1995), different species of which can comprise the formulations of the inventions, as well as components of the invented molecules; p120 (Schreier, et al., J. Biol. Chem., 269: 9090, 1994); See also Keinanen, et al., FEBS Lett., 346: 123, 1994; Killion, et al., Immunomethods, 4: 273, 1994. In some methods, the therapeutic compounds of the invention are formulated in liposomes; in a more preferred aspect, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

### M. Formulation

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

Bcl proteins for oral administration can be formulated using pharmaceutically acceptable carriers well known in the art, in dosages suitable for oral administration. Such carriers enable the Bcl proteins to be formulated as tablets, pills, dragees, capsules, liquids, gels, syrups, slurries, suspensions, etc., suitable for ingestion by a subject.

Bcl proteins, which can be used orally, can be formulated, for example, as push-fit capsules made of gelatin, as well as soft, sealed capsules made of gelatin and a coating such as glycerol or sorbitol. Push-fit capsules can contain Bcl proteins mixed with filler or binders such as lactose or starches, lubricants such as talc or magnesium stearate, and, optionally, stabilizers. In soft capsules, the Bcl proteins can be dissolved or suspended in suitable liquids, such as fatty oils, liquid paraffin, or liquid polyethylene glycol with or without stabilizers.

Bcl proteins for oral use can be obtained, for example, through combination of one or more Bcl proteins with solid excipient, optionally grinding the resulting mixture, and processing the mixture of granules, after adding suitable additional compounds, if desired, to obtain tablets or dragee cores. Suitable excipients include carbohydrate or protein fillers. These include, but are not limited to, sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilizing agents can be added, such as the cross-linked polyvinyl pyrrolidone, agar, alginic acid, or a salt thereof, such as sodium alginate.

Dragee cores are provided with suitable coatings such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound (i.e., dosage).

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

### N. Kits

In another aspect, there are one or more kits for making and/or using the methods and reagents of the invention. The components of the kit are housed in a suitable container and can be sterile, where appropriate. Kit housing can include boxes, vials, or bottles, for example.

After the Bcl proteins of the invention are formulated in an acceptable carrier have been prepared as described above, they can be placed in an appropriate container and labeled for use to reduce the incidence, extent or severity of cell death or inflammation in a mammal comprising administering to the mammal concurrent with or after the onset of a condition expected to lead to cell death or inflammation an amount of a Bcl protein effective to attain said reduction.

In other aspects, there are components for application of the Bcl therapeutic proteins to an individual, such as a syringe, a filter, an aqueous solution, a needle, a syringe, and so forth. A therapeutic product can include sterile saline or another pharmaceutically acceptable emulsion and suspension base as described above.

Kits of the present invention can also contain additional agents that can be administered concomitantly with the compounds of the present invention. In addition, kits can contain reagents or other components (e.g., the Bcl protein can also be administered with at least one additional agent).

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The following Exemplary Aspects of specific aspects for carrying out the present invention are offered for illustrative purposes only, and are not intended to limit the scope of the present invention in any way.

### EXEMPLARY ASPECTS

### Example 1

This example describes expression of a cDNA (SEQ ID NO:13) that encodes the anti-apoptotic protein, human Bcl-2 (SEQ ID NO:14) in myeloid cells. Expression of the Bcl-2 protein (SEQ ID NO:14) in myeloid cells reduced injury following extended ischemia in the mouse hind limb.

Mice expressing a recombinant human Bcl-2 (SEQ ID NO:14) in their myeloid cells (hMRP8-myeloid-Bcl-2 mice), and control mice that did not express the recombinant human Bcl-2 (SEQ ID NO:14) in their myeloid cells were used in this experiment. The hMRP8-myeloid-Bcl-2 mice were previously described by Lagasse, E., and I. L. Weissman, J. Exp. Med. 179:1047, 1994, which publication is incorporated herein by reference. These mice were on a C57BL/6 background, and the control mice were C57BL/6 mice.

Mouse skeletal muscle was made ischemic by cross-clamping the aorta distal to the renal artery for 90 minutes, then the clamp was removed and hind limb reperfusion continued for 3 hours. At the end of reperfusion (3 hours after clamp removal) the mice were killed, and the concentration of plasma creatine kinase (CK) was measured and used as an indicator of injury (creatine kinase concentration increases as a result of skeletal muscle injury).

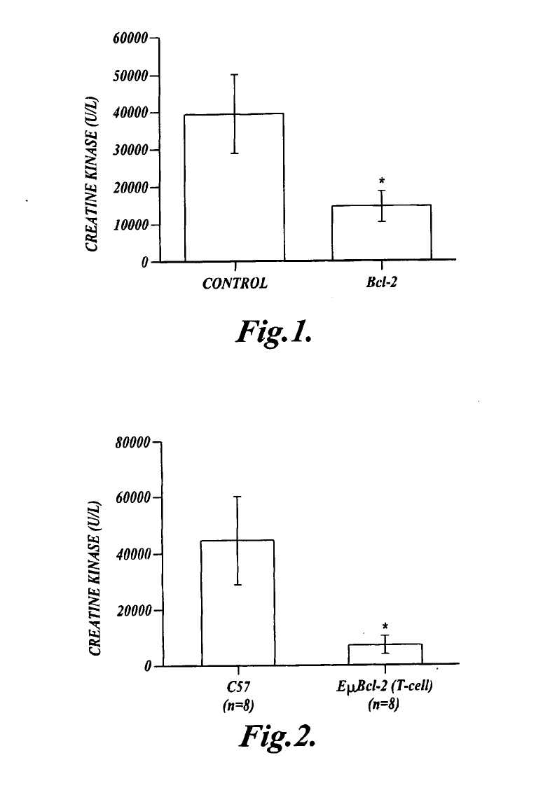

The results of these experiments are shown in FIG. 1. The plasma creatine kinase levels were significantly less in the eleven hMRP8-myeloid-Bcl-2 mice (designated Bcl-2) compared to the nine control C57BL/6 mice (*p<0.05), suggesting that human Bcl-2 (SEQ ID NO:14) protects the mice from ischemia-reperfusion injury. It has been reported in the literature, however, that neutrophils from the hMRP8-myeloid-Bcl-2 mice exhibit reduced apoptosis (Lagasse, E., and Weissman, I. L., J. Exp. Med. 179:1047, 1994), and it is possible that neutrophil apoptosis contributes to ischemia-reperfusion injury by release of toxic products at the site of injury. Thus, it is possible that Bcl-2 is preventing apoptosis of neutrophils, thereby reducing the amount of ischemia-reperfusion injury in the mice that express Bcl-2 in their myeloid cells.

It is unlikely, however, that neutrophils are involved in the ischemia-reperfusion injury for the extended ischemia time of 90 minutes since blocking their emigration into tissue with anti-CD18 mAb has no effect on ischemia-reperfusion injury (Iwata, A. et al., Blood, 100:2077, 2002). Moreover, as shown in Example 2, over-expression of human Bcl-2 (SEQ ID NO:13) in T-lymphocytes also protects against ischemia-reperfusion injury. Thus, it appears to be unlikely that over-expression of human Bcl-2 in myeloid cells is protecting muscle by preventing apoptosis of leukocytes.

### Example 2

This example shows that over-expression of human Bcl-2 (SEQ ID NO:14) in T-lymphocytes reduces skeletal muscle injury following extended ischemia in the mouse hind limb.

Transgenic mice, on a C57BL/6 genetic background, expressing exogenous human Bcl-2 (SEQ ID NO:14) in their T-cells under the control of the Eμ-promoter (EμT-Bcl-2 mice), and eight C57BL/6 control mice that did not express exogenous Bcl-2 (SEQ ID NO:14) in their T-cells (C57BL/6 mice) were used in this experiment. The EμT-Bcl-2 mice have been previously described and shown to express Bcl-2 (SEQ ID NO:14) only in T-lymphocytes (Strasser, A., et al., Cell 67:889, 1991, which publication is incorporated herein by reference).

Hind limb ischemia was induced by cross clamping the aorta as described in Example 1, and ischemia was maintained for 90 minutes followed by 3 hours of reperfusion. Blood samples were taken at the end of the experiment for determination of creatine kinase (CK) concentration in EμT-Bcl-2 mice and C57Bl/6 mice. As shown in FIG. 2, serum CK in the eight EμT-Bcl-2 mice was significantly less than in the eight C57BL/6 control mice (designated C57) (*p<0.05).

### Example 3

This example shows that over-expression of Bcl-2 (SEQ ID NO:14) in leukocytes reduces DNA strand-breaks in skeletal muscle following extended ischemia and reperfusion of the hind limb.

Transgenic mice (described in Example 2) expressing exogenous human Bcl-2 (SEQ ID NO:14) in their T-cells under the control of the Eμ-promoter (EμT-Bcl-2 mice); transgenic mice expressing exogenous human Bcl-2 (SEQ ID NO:14) in their B-cells under the control of the Eμ-promoter (EμB-Bcl-2 mice) (reported in Strasser, A., Proc. Natl. Acad. Sri. 88:8661, 1991); transgenic mice (described in Example 1) expressing exogenous human Bcl-2 (SEQ ID NO:14) in their myeloid cells (hMRP8-myeloid-Bcl-2 mice); and control mice (C57BL/6 mice) that did not express exogenous Bcl-2 (SEQ ID NO:14), were used in this experiment.

It is known that extended ischemia followed by reperfusion of skeletal muscle results in DNA strand-breaks, and that treatment with the caspase inhibitor z-VAD prevents the strand-breaks and reduces the plasma CK concentration (Iwata, A., et al., Blood 100:2077, 2002). Tissue from the legs of control mice, EμT-Bcl-2 mice, EμB-Bcl-2 mice and hMRP8-myeloid-Bcl-2 transgenic mice was fixed in formalin and stained to identify DNA strand breaks using the TUNEL technique as described by the manufacturer (In Situ Cell Death Detection Kit, Roche Applied Science, PO Box 50414, 9115 Hague Road, Indianapolis, Ind. 46250-0414).

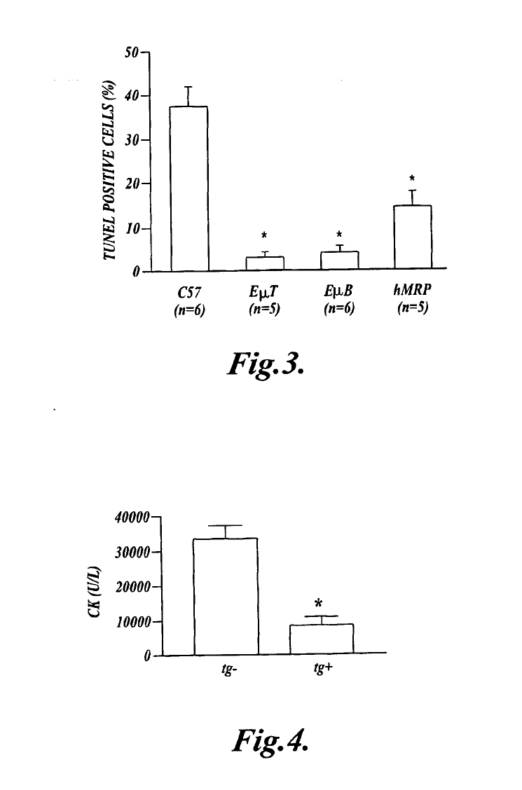

The number of nuclei that stained positive (indicating the presence of DNA strand breaks) as a percent of the total number of nuclei is shown in FIG. 3 for all four types of mice. The control C57BL/6 mice (designated C57) had significantly more positive nuclei compared with each of the transgenic strains (*p<0.05). In the mice that expressed Bcl-2 (SEQ ID NO:14) in one cell type (EμB in B cells, EμT in T cells, or hMRP8 in myeloid cells) DNA strand breaks were prevented in skeletal muscle and endothelial cells, other than the cells that expressed the Bcl-2 (SEQ ID NO:14), suggesting that the cells that expressed Bcl-2 released a molecule that protected cells from DNA strand breaks. That is, protection occurs as a “trans” effect.

### Example 4

This example shows that blood plasma from mice over-expressing hBcl-2 (SEQ ID NO:14) in T-lymphocytes reduces injury following extended ischemia followed by reperfusion.

Transgenic mice (described in Example 2) expressing exogenous human Bcl-2 (SEQ ID NO:14) in their T-lymphocytes (EμT-Bcl-2 mice), and littermate control mice that did not express exogenous human Bcl-2 (SEQ ID NO:14) (C57Bl/6 mice) were used in this experiment. Blood from EμT-Bcl-2 mice and their littermate control mice was drawn into heparin and plasma was extracted by centrifugation. One ml of plasma from EμT-Bcl-2 mice, or one ml of plasma from littermate control mice, was injected into the peritoneum of C57BL/6 mice the day before the mice were subjected to 90 minutes of ischemia and 3 hours of reperfusion as described in Example 1 herein. Blood was drawn from the mice and plasma CK concentration was determined at the end of reperfusion. The results of these experiments are shown in FIG. 4. The six mice that received an injection of plasma from the EμT-Bcl-2 mice (designated tg+) had significantly lower concentrations of CK compared with the six mice that received an injection of plasma from littermate control mice (designated tg−) that did not express exogenous Bcl-2 (SEQ ID NO:14) (*p<0.05).

These results show that over-expression of Bcl-2 (SEQ ID NO:14) in the hMRP8-Bcl-2 mice results in the release of a molecule, that acts in “trans”, that can be transferred to naive, control, recipient mice and that protects the recipient mice from ischemia-reperfusion injury.

### Example 5

This example shows that plasma creatine kinase levels in mice that had been subjected to hind leg ischemia and reperfusion was significantly lower in mice that were injected with modified, recombinant, Bcl-2 (SEQ ID NO:15) before hind leg ischemia and reperfusion, compared to the plasma creatine kinase levels in mice that were not injected with recombinant Bcl-2 before hind leg ischemia and reperfusion. SEQ ID NO:15 is a human Bcl-2 that lacks 17 amino acids at the carboxy terminal, and includes a series of 10 histidine residues on the carboxy terminal.

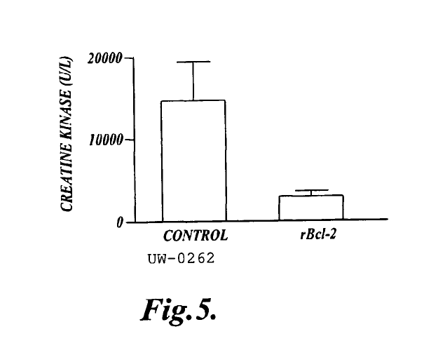

C57BL/6 mice were injected intraperitoneally with 1 μg per mouse of recombinant human Bcl-2 (rBcl-2) (SEQ ID NO:15) or 1 μg per mouse of recombinant human ubiquitin (rUbiquitin), or the vehicle solution for rBcl-2 the day before the mice were subjected to hind limb ischemia (90 minutes) and reperfusion (180 minutes) as described in Example 1. Blood samples were taken after the 90 minutes of hind limb ischemia and 180 minutes of reperfusion for determination of plasma creatine kinase concentration. There was no difference in the creatine kinase concentration between the two controls (rUbiquitin and vehicle solution) and these data were combined. As shown in FIG. 5, the plasma creatine kinase levels in mice that had been subjected to hind leg ischemia and reperfusion were significantly (p<0.05) lower in the 12 mice that were injected with recombinant human Bcl-2 (SEQ ID NO:15) (designated rBcl-2) before hind leg ischemia and reperfusion, compared to the plasma creatine kinase levels in the 12 mice that received rUbiquitin or vehicle solution (designated CONTROL).

### Example 6

This example shows that over-expression of human Bcl-2 (SEQ ID NO:14) under a myeloid-restricted promoter reduces cardiomyocyte injury following extended ischemia in the mouse heart.

In order to determine whether over-expression of a Bcl-2 protein in leukocytes was protective in tissue other than skeletal muscle, myocardial ischemia-reperfusion injury was examined using ischemia times that were known to be CD18-independent (Palazzo, A. J., et al., Am. J. Physiol. 275:H2300, 1998). Control C57BL/6 and hMRP8-Bcl-2 mice were anesthetized, their trachea intubated, and they were placed on mechanical ventilation. A left thoracotomy was performed then an 8-0 suture was passed under the left anterior descending coronary artery (LAD) 2-3 mm from the tip of the left auricle, and the vessel was occluded. Care was taken not to damage the vessel. Occlusion was confirmed visually by change in color. The ligature was carefully removed after 1 hour of occlusion and reperfusion verified by direct visualization as color was re-established. The chest was closed taking care to remove air from the chest, the animal was extubated, given 0.5 ml of warmed saline, and placed in a heated incubator. Two hours later the mice were re-anesthetized, their trachea intubated, and they were placed on mechanical ventilation. The heart was exposed through the original incision and the original 8-0 suture re-tied. The mice were killed by exsanguination and a clamp was placed across the aorta, then 1 ml of 1.5% Evans Blue dye was injected by inserting a 30 gauge needle into the aorta so that the coronary circulation was perfused with dye.

The heart was removed, cut perpendicular to the long axis resulting in 4 sections that were incubated in 5 ml of 1% triphenyltetrazolium chloride (TTC) for 30 minutes. The left ventricle was placed in 10% buffered formaldehyde solution overnight following the removal of both the atrium and the right ventricle. Each heart slice was weighed then visualized under a microscope equipped with a CCD camera. The infarct area (uncolored), area at risk (AAR) (uncolored region plus brick red region) and total left ventricular region (AAR plus Evans Blue stained region) were measured by planimetry. The volume of infarction was estimated by the following equation:

V_{infarct}=A₁W₁+A₂W₂+A₃W₃+A₄W₄

Where A1, A2, A3, and A4 are the percent area of infarction in section 1, 2, 3, and 4, respectively and W1, W2, W3, and W4 are the corresponding weight in section 1, 2, 3, and 4, respectively. The volume at risk was calculated in a similar manner using appropriate areas.

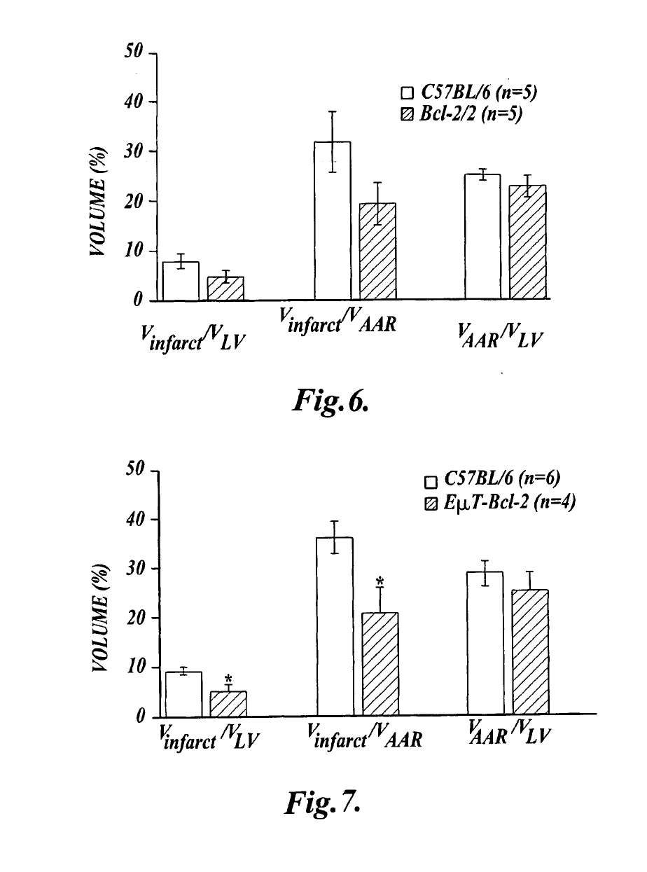

FIG. 6 shows the infarct volume as a percentage of left ventricular volume and as a percentage of the area at risk volume. The five hMRP8-Bcl-2 mice (designated Bcl-2/2) had reduced infarct volume by both measures compared with the five C57BL/6 control mice, and there were no differences in volume at risk to left ventricular volume between these two groups. V_{infarct}/V_{LV} and V_{infarct}/V_{AAR} of hMRP8-Bcl-2 mice were significantly reduced compared to C57BL/6 (p<0.05). There was no difference in volume at risk to left ventricular volume (V_{AAR}/V_{LV}) between the two groups.

### Example 7

This example shows that transgenic mice that express exogenous human Bcl-2 (SEQ ID NO:14) in their T-lymphocytes suffer less cardiomyocyte damage, caused by ischemia followed by reperfusion, than control mice that do not express exogenous Bcl-2 protein (SEQ ID NO:14) in their T-lymphocytes.

Additional myocardial ischemia-reperfusion experiments were performed using EμT-Bcl-2 mice that over-expressed Bcl-2 (SEQ ID NO:14) in their T-lymphocytes under the control of the Eμ promoter, and C57BL/6 control mice. The experiments were performed as described in Example 6, with coronary artery occlusion for 1 hour followed by 2 hours of reperfusion. Infarct volume (V_{infarct}) was calculated as a percent of left ventricle volume (V_{LV}), or as a percent of the volume of the area at risk (VAAR). As shown in FIG. 7, V_{infarct}/VLV and V_{infarct}/V_{AAR} were significantly reduced in the Eμ-T-lymphocyte-Bcl-2 mice versus C57BL/6 mice. (*p<0.05). There was no difference in volume at risk to left ventricular volume (V_{AAR}/V_{LV}) between the two groups.

### Example 8

This example shows that adoptive transfer of myeloid cells that express exogenous Bcl-2 protein (SEQ ID NO:14) reduces cardiomyocyte injury following extended ischemia in the mouse heart.

hMRP8-myeloid-Bcl-2 mice and littermate control mice were anesthetized, killed and bone marrow was extracted from their long bones. CD11b+ cells in the extracted bone marrow were isolated using magnetic beads (Miltenyi Biotec, 12740 Earhart Avenue, Auburn, Calif. 95602, USA) as described by the manufacturer. Approximately 10⁷ of these cells were administered to C57BL/6 control mice by intra-peritoneal injection 18 to 24 hours prior to hind limb ischemia and reperfusion. The ischemic period was 1 hour followed by 2 hours of reperfusion. Determination of infarct size was completed using the same technique as described in Example 6.

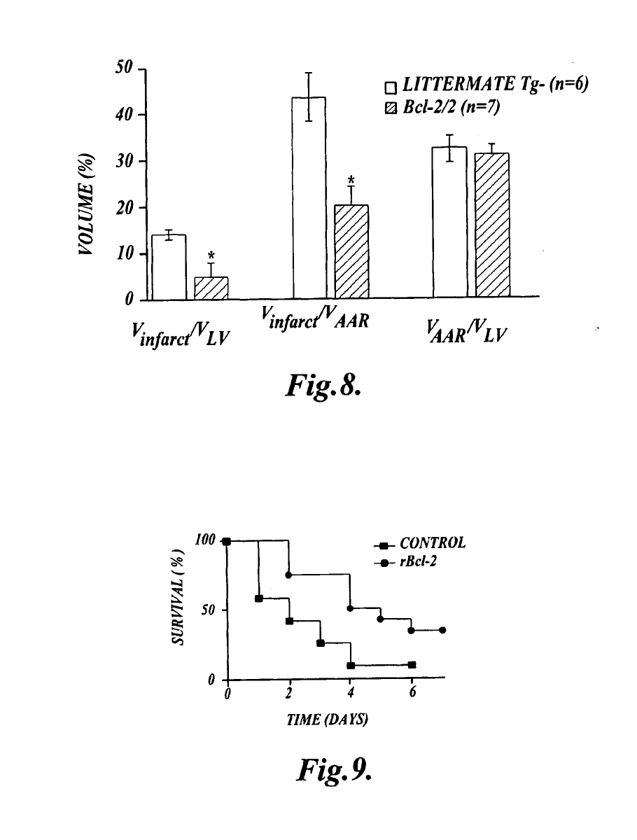

FIG. 8 shows the infarct volume as a percentage of left ventricular volume and as a percentage of the area at risk volume. The seven mice receiving bone marrow cells from the hMRP8-myeloid-Bcl-2 mice (designated Bcl-2/2) had significantly (*p<0.05) reduced infarct volume by both measures (V_{infarct}/V_{LV} and V_{infarct}/V_{AAR}) compared with the six mice that received bone marrow cells from littermates (designated littermate Tg−). There was no difference in volume at risk to left ventricular volume (V_{AAR}/V_{LV}) between the two groups.

### Example 9

This example shows that over-expression of Bcl-2 provides protection in septic mice by a “trans” effect.

The survival of transgenic mice that expressed exogenous Bcl-2 (SEQ ID NO:14) in myeloid cells, under control of the human MRP8 promoter (hMRP8-Bcl-2 mice), or in T lymphocytes, under control of the Eμ promoter (EμT-Bcl-2 mice), was compared with the survival of C57BL/6 control mice following cecal ligation and puncture (CLP). 100% of hMRP8-Bcl-2 mice survived CLP, whereas only 25% of control mice survived CLP (p<0.05). In a separate experiment, 87.5% of EμT-Bcl-2 mice survived CLP, whereas only 22.2% of control mice survived CLP (p<0.05).

CD11b-positive bone marrow cells from hMRP8-Bcl-2 mice, or from C57BL/6 mice, were introduced into C57BL/6 mice, which were then subjected to CLP. 100% of the mice that received CD11b-positive bone marrow cells from hMRP8-Bcl-2 mice survived CLP, while none of the mice that received CD11b-positive bone marrow cells from C57BL/6 mice survived CLP.

CD11b-positive bone marrow cells from hMRP8-Bcl-2 mice, or from C57BL/6 mice, were introduced into Rag-1−/− mice (that did not have any mature T or B cells), which were then subjected to CLP. 87.5% of the mice that received CD11b-positive bone marrow cells from hMRP8-Bcl-2 mice survived CLP, while 12.5% of the mice that received CD11b-positive bone marrow cells from C57BL/6 mice survived CLP (p<0.05).

These experiments show that expression of hBcl-2 (SEQ ID NO:14) is protective in CLP and that protection is independent of lymphocytes.

### Example 10

This example shows that intraperitoneal injection of recombinant human Bcl-2 (rhBcl-2) (SEQ ID NO:15) prior to injury improves survival in mice subjected to severe sepsis as a result of cecal ligation and puncture.

Eight C57BL/6 mice were given an intraperitoneal injection of 1 μg per mouse rhBcl-2 and eight C57BL/6 mice were given and intraperitoneal injection of 1 μg per mouse recombinant human ubiquitin (rhUbiquitin), an unrelated protein generated by similar recombinant technology as rhBcl-2, 12-24 hours prior to being subjected to cecal ligation and puncture as described in Example 9. An additional four C57BL/6 mice were given a subcutaneous injection of 10 μg of a maltose binding protein-hBcl-2 fusion protein and four C57BL/6 mice were given saline treatment 12-24 hours prior to being subjected to cecal ligation and puncture as described in Example 9. The mice were treated with antibiotics twice daily and with an additional treatment of recombinant human Bcl-2 or recombinant human ubiquitin or saline given daily for 3 days. Examination was conducted for signs of irreversible sepsis twice daily for 10 days through the use of a quantitative assessment form, and the mice were killed if they were deemed to be suffering from irreversible sepsis. FIG. 9 is a survival curve based on the results of these experiments and clearly shows that the 12 mice pretreated with recombinant human Bcl-2 (designated rBcl-2) had significantly (p<0.05) improved survival compared to the 12 mice treated with either recombinant human ubiquitin or saline (designated CONTROL).

### Example 11

This example shows that plasma creatine kinase levels in mice that had been subjected to hind leg ischemia and reperfusion was significantly lower in mice that were pretreated with modified recombinant human A1/Bfl-1/Bfl-1 (rhA1/Bfl-1; human A1 minus 25 amino acids at the carboxy terminal and addition of 6 histidine on the remaining protein) (SEQ ID NO:16) before hind leg ischemia and reperfusion, compared to the plasma creatine kinase levels in mice that were injected with recombinant ubiquitin before ischemia and reperfusion. Recombinant human A1 (SEQ ID NO:16) is an anti-apoptotic Bcl-2 protein with a BH4-like domain in the first alpha helix (Class III Bcl-2 protein (Zhang et al, J Biol Chem, 275:11092, 2000).

C57BL/6 mice were injected intraperitoneally with rhA1/Bfl-1 (1 microgram/mouse) (SEQ ID NO:16) or rhUbiquitin (1 microgram/mouse) the day before the mice were subjected to hind limb ischemia (90 minutes) and reperfusion (180 minutes) as described in Example 1. Blood samples were taken after the 180 minutes of reperfusion for determination of plasma creatine kinase concentration. The average plasma creatine kinase concentration in mice treated with rhA1/Bfl-1 was 4,353+/−1284 IU/L and plasma creatine kinase concentration in mice treated with rhUbiquitin was 22,990+/−7256 IU/L. The plasma creatine kinase concentration in mice that had been subjected to hind leg ischemia and reperfusion were significantly (p<0.05) lower in the 12 mice that were injected with rA1 (SEQ ID NO:16) before hind leg ischemia and reperfusion, compared to the plasma creatine kinase levels in 12 control mice that were injected with rUbiquitin.

### Example 12

This example shows that pretreatment with fragments of a BH4 domain or BH4-like domain protected the hind-limbs of C57BL/6 mice from ischemia-reperfusion injury.

Skeletal muscle was made ischemic by applying a tourniquet to the hind-limbs of C57BL/6 mice for 90 minutes, then removing the tourniquet and allowing reperfusion for an additional 3 hours. Mice were treated with active peptide 1 (SEQ ID NO:17) or peptide 2 (SEQ ID NO:18) or peptide 3 (SEQ ID NO:19), or with the scrambled (control) peptide (SEQ ID NO:20). Peptide-1 (SEQ ID NO:17) is from the BH4 region of Bcl-2. Peptide-2 (SEQ ID NO:18) is from the first alpha helix of A1/Bfl-1. Peptide-3 (SEQ ID NO:19) is from the BH4 region of Bcl-XL. The amino acid sequences of peptide 1 (SEQ ID NO:17), peptide 2 (SEQ ID NO:18), peptide 3 (SEQ ID NO:19), and the scrambled (control) peptide (SEQ ID NO:20) are set forth in Table 1.

TABLE 1

| | | |
|---|---|---|
| TGYDNREIVMKYIHYKLSQRGYEWD | peptide-1 | (SEQ ID NO: 17) |
| FGYIYRLAQDYLQCVLQIPZPGSGP | peptide-2 | (SEQ ID NO: 18) |
| MSQSNRELVVDFLSYKLSQKGYSWS | peptide-3 | (SEQ ID NO: 19) |
| QF | | |
| TWHMYGNQRDYIGDRSKIVYKLEYE | scrambled | (SEQ ID NO: 20) |
| | peptide | |

At the end of the 3 hours of reperfusion the mice were killed, and blood was taken for determination of plasma creatine kinase (CK) concentration. The CK concentration was used as an indicator of muscle injury. Elevated levels of CK indicate higher levels of muscle injury. Results from 3 separate experiments showed significant protection compared with the control peptide (p<0.05). The CK concentration for peptide 1 (SEQ ID NO:17) treated mice was 19,020+/−5481 IU/L (n=12) compared with control peptide (SEQ ID NO:20) where CK concentration was 60,530+/−5759 IU/L (n=12). The CK concentration for peptide 2 (SEQ ID NO:18) treated mice was 33,860+/−5997 IU/L (n=11) compared with control peptide (SEQ ID NO:20) where CK concentration was 69,430+/−11,170 IU/L (n=12). The CK concentration for peptide 3 (SEQ ID NO:19) treated mice was 49,500+/−3,901 IU/L (n=5) compared with control peptide (SEQ ID NO:20) where CK concentration was 80,880+/−11,430 IU/L (n=6).

### Example 13

This example shows that administration of rhA1/Bfl-1 or rhBcl2 at the time of (concurrent treatment) or after (post-treatment) reperfusion following ischemia increases viability of tissue subjected to ischemia-reperfusion.

Anesthetized mice were subjected to 90 minutes of tourniquet ischemia using a hemorrhoid ligator followed by reperfusion and were treated at various times by a subcutaneous injection of 1 microgram of either rhA1/Bfl-1 or rhBim, a pro-apoptotic Bcl-2 family member that lacks a BH4 or BH4-like domain (O'Connor et al., EMBO J. 17:384, 1998). The following day, mice were killed, the previously ischemic muscle removed, and tissue viability measured using 3-(4,5-dimethylthiazol-2-yl)-2,5-diphenyl tetrazolium (MTT). Briefly, the excised muscle tissue was cut into pieces to increase the surface area and uptake of MTT. The tissue sections were placed in 3 ml of PBS supplemented with 60 micro liters of 5 mg/ml MTT and incubated for 3 hours at 37° C. on a shaker. Samples were removed, blotted dry and the insoluble formazam salt extracted in 3 ml of 2-propanol overnight at 37° C. in the dark. Absorbance at 570 nm of 200 microliters of the solution was determined and normalized to the dry weight of the tissue. Viable mitochondria reduce the MTT to an insoluble salt that is extracted from cells and is soluble in propanol. Thus, live tissue has an increased optical density relative to dead.

Treatment at Reperfusion.

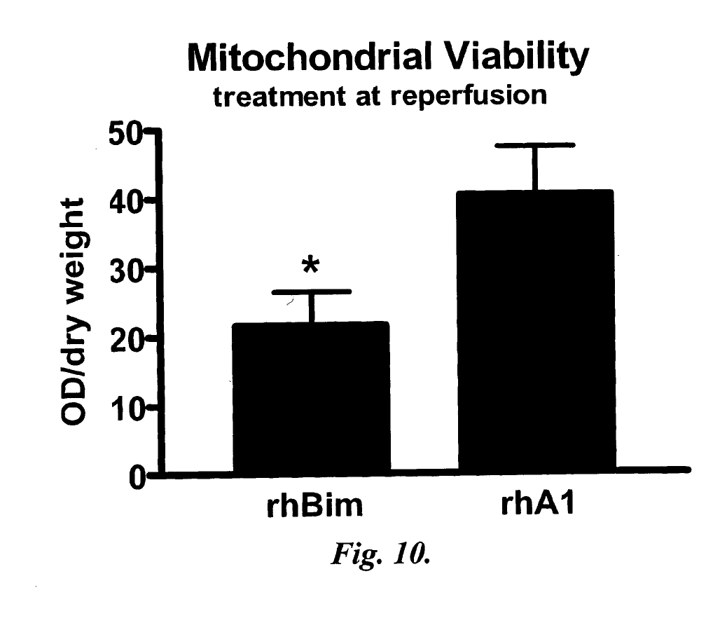

Mice were subjected to ischemia/reperfusion and tissue viability measured as described above. Viability of the tissue normalized to tissue dry weight from mice treated at the time of reperfusion is shown in FIG. 10. Clearly, tissue from mice treated with rhA1/Bfl-1 showed greater viability than mice treated with the control protein rhBim. These experiments demonstrated that rhA1/Bfl-1 is effective when administered at the time of reperfusion.

Treatment 4 Hours Post Reperfusion.

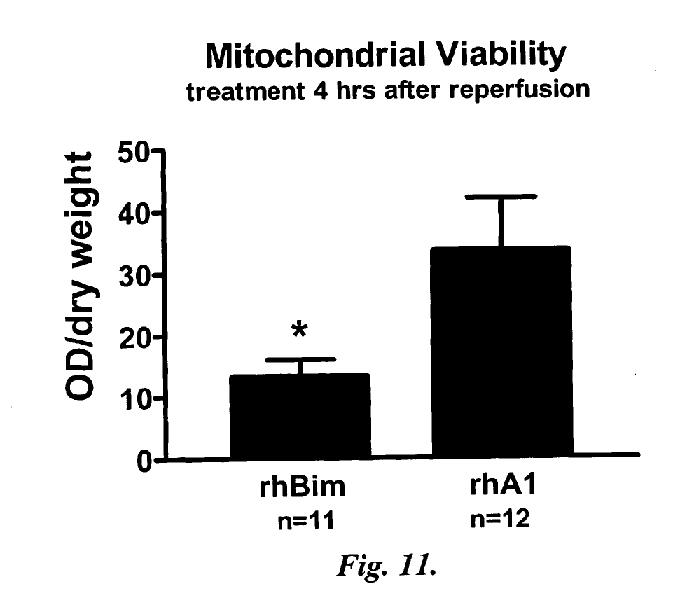

Mice were subjected to ischemia/reperfusion and tissue viability measured as described above. Tissue viability normalized to tissue dry weight from mice treated 4 hours after the start of reperfusion is shown in FIG. 11. The rhA 1/Bfl-1 treated mice had significantly greater tissue viability compared with rhBim-treated mice. These experiments demonstrate that rhA1/Bfl-1 is effective in treating I/R injury even treatment is delayed until after reperfusion of the ischemic tissue.

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

1. A method to reduce the incidence, extent or severity of cell death or inflammation in a mammal comprising administering to the mammal after the onset of a condition expected to lead to cell death or inflammation an amount of a Bcl protein effective to attain said reduction.

2. The method of claim 1 wherein the Bcl protein is administered to the mammal up to 48 hours after the onset of a condition expected to lead to cell death or inflammation.

3. The method of claim 1 wherein the Bcl protein is administered to the mammal up to 24 hours after the onset of a condition expected to lead to cell death or inflammation.

4.-15. (canceled)

16. The method of claim 1 wherein the Bcl protein is administered to the mammal intermittently or continuously for at least 24 hours from the onset of a condition expected to lead to cell death or inflammation.

17. The method of claim 1 wherein the Bcl protein is administered to the mammal intermittently or continuously for at least 12 hours from the onset of a condition expected to lead to cell death or inflammation.

18. The method of claim 1 wherein the Bcl protein is administered to the mammal in combination with at least one additional agent.

19. The method of claim 1, wherein the Bcl protein is a sterile pharmaceutical formulation.

20.-24. (canceled)

25. The method of claim 1, wherein the mammal is a human being.

26. The method of claim 1, wherein the Bcl protein is administered intravenously.

27.-29. (canceled)

30. The method of claim 1, wherein the Bcl protein is
a protein comprising at least 12 amino acids, wherein the protein is at least 50% similar to a segment of an A1/Bfl-1 protein, wherein the A1/Bfl-1 protein consists of the amino acid sequence set forth in SEQ ID NO:4.

31.-32. (canceled)

33. A method of claim 1, wherein the Bcl protein is administered to the mammalian subject in an amount from 0.5 μg/kg/day to 50 μg/kg/day for a period of time sufficient to effect attain said reduction.

34. (canceled)

35. A method of claim 1, wherein the Bcl protein is administered to the mammalian subject daily.

36. A method to reduce the incidence, extent or severity of cell death or inflammation in a mammal comprising administering to the mammal concurrent with the onset of a condition expected to lead to cell death or inflammation an amount of a Bcl protein effective to attain said reduction.

37. The method of claim 36 wherein the Bcl protein is administered to the mammal in combination with at least one additional agent.

38. The method of claim 36, wherein the Bcl protein is a sterile pharmaceutical formulation.

39.-43. (canceled)

44. The method of claim 36, wherein the mammal is a human being.

45. The method of claim 36, wherein the Bcl protein is administered intravenously.

46.-48. (canceled)

49. The method of claim 36, wherein the Bcl protein is
a protein comprising at least 12 amino acids, wherein the protein is at least 50% similar to a segment of an A1/Bfl-1 (BCL2A1) protein, wherein the A1/Bfl-1 (BCL2A1) protein consists of the amino acid sequence set forth in SEQ ID NO:4.

50. (canceled)

51. A method of claim 36, wherein the Bcl protein is administered to the mammalian subject in an amount from 0.5 μg/kg/day to 50 μg/kg/day for a period of time sufficient to effect attain said reduction.

52. (canceled)

53. A method for inhibiting inflammation in a mammal, the method comprising the step of administering to a mammal concurrent with or after the onset of inflammation, a Bcl protein in an amount sufficient to inhibit a inflammation in the mammal.
